(19) United States
(12) Patent Application Publication
(10) Pub. No.: US 2023/0416243 A1
(43) Pub. Date: Dec. 28, 2023

(54) Process for Preparing Heterocyclic Methanone Compounds and AZA-Bicyclo Intermediates Thereof

(71) Applicant: ACTINOGEN MEDICAL LIMITED, Sydney, New South Wales (AU)

(72) Inventor: Christian GRUGEL, New South Wales (AU)

(21) Appl. No.: 18/252,025

(22) PCT Filed: Nov. 5, 2021
(86) PCT No.: PCT/AU2021/051310

§ 371 Date: May 5, 2023

(30) Foreign Application Priority Data
Nov. 6, 2020 (AU) 2020904060

(51) Int. Cl. C07D 451/06

(52) U.S. Cl. CPC C07D 451/06

(57) ABSTRACT

The present disclosure relates to a process for synthesis of heterocyclic methanone compounds, and in particular 3′-substituted, 3-hydroxyl-(8-aza-bicyclo[3.2.1]oct-8-yl)-[5-(1h-pyrazol-4-yl)-thiophen-3-yl]-methanone compounds, and aza-bicyclo intermediates thereof. In particular, the present disclosure also relates to a process for the synthesis of Xanamem. The present disclosure also relates to a process for the synthesis of optionally protected aza-bicyclo intermediate compounds. The present disclosure also relates to 3′-substituted, 3-hydroxyl-(8-aza-bicyclo[3.2.1]oct-8-yl)-[5-(1h-pyrazol-4-yl)-thiophen-3-yl]-methanone compounds and aza-bicyclo intermediate compounds thereof.

### FIELD

The present disclosure generally relates to a process for synthesis of heterocyclic methanone compounds, and in particular 3′-substituted, 3-hydroxyl-(8-aza-bicyclo[3.2.1]oct-8-yl)-[5-(1h-pyrazol-4-yl)-thiophen-3-yl]-methanone compounds, and aza-bicyclo intermediates thereof. In particular, the present disclosure also relates to a process for the synthesis of Xanamem. The present disclosure also relates to a process for the synthesis of optionally protected aza-bicyclo intermediate compounds. The present disclosure also relates to 3′-substituted, 3-hydroxyl-(8-aza-bicyclo[3.2.1]oct-8-yl)-[5-(1h-pyrazol-4-yl)-thiophen-3-yl]-methanone compounds and aza-bicyclo intermediate compounds thereof, which have been prepared by any of the processes of the present disclosure. The present disclosure also relates to pharmaceutical compositions comprising the 3′-substituted, 3-hydroxyl-(8-aza-bicyclo[3.2.1]oct-8-yl)-[5-(1h-pyrazol-4-yl)-thiophen-3-yl]-methanone compounds, and in particular Xanamem.

### BACKGROUND

Xanamem, also known as UE2343, is an effective inhibitor of 11β-hydroxysteroid dehydrogenase type 1 (11β-HSD1). Due to its inhibitory action and associated reduction of cortisol levels, Xanamem has been proposed as a treatment of Alzheimer's disease.

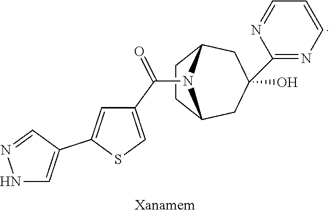 [SMILES: O=C(c1csc(-c2cn[nH]c2)c1)N1[C@@H]2CC[C@H]1C[C@](O)(c1ncccn1)C2]

To date, the only reported process for preparing Xanamem comes from the international PCT publication WO2011135276. According to the reported preparation process, a carboxylic acid derivative (left-hand side) and a amine bicyclic derivative (right-hand side) of the molecule are separately synthesised, before a final amide coupling reaction is undertaken using 1-[bis(dimethyl amino)methylene]-1H-1,2,3-triazolo[4,5-b]pyridinium 3-oxide hexafluorophosphateto (HATU) coupling reagent in dichloromethane to yield Xanamem.

A particular drawback to the reported process identified by the present inventors lies in the synthesis of the right-hand section of Xanamem, and particularly the coupling of the pyrimidine moiety with the nortropinone moiety. The reaction involves the use of the highly reactive, pyrophoric reagent, n-butyllithium. As a consequence, the reaction must be carefully maintained at cryogenic temperatures, particularly −95° C. The addition of n-butyllithium to the reaction mixture results in an exothermic reaction, thereby increasing the temperature of the reaction mixture upon its addition. The reaction therefore requires the slow addition of n-butyllithium and the careful monitoring of the reaction temperature throughout the addition of the n-butyllithium. While such a reaction may be suitable for small-scale synthesis, the reaction does not lend itself to a scale-up process for preparing larger quantities of Xanamem.

Another particular drawback the present inventors identified with respect to the reported process lies in the final amide coupling reaction of the thiophene carboxylic acid and the nortropinone amine. The reaction, which utilises a HATU coupling reagent, results in tetramethylurea (TMU) as a by-product, which is itself a potential genotoxic compound. Even further, this TMU by-product is difficult to separate from Xanamem during purification. Again, while such a reaction and related post reaction purification steps may be suitable for the small-scale synthesis of Xanamem, this reaction is not suitable to a scale-up process for preparing larger quantities of Xanamem.

Accordingly, there remains a need for a safe, efficient, and scalable synthesis of Xanamem and related analogues, along with high purity where the generation of any undesirable by-products are significantly reduced or avoided.

### SUMMARY

The subject matter of the present disclosure is predicated in part on the surprising discovery that the utilisation of Grignard reaction conditions for the reaction system can obviate the need for cryogenic reaction conditions in preparing aza-bicyclo intermediate compounds, and/or specific amide coupling reaction conditions for preparing heterocyclic methanone compounds can avoid the genotoxic tetramethylurea (TMU) by-product, also resulting in an efficient and scalable synthesis of Xanamem.

The present disclosure also relates to a process for preparing aza-bicyclo compounds comprising a Grignard reaction of a nortropinone compound with a halogenated compound. The present disclosure also relates to a process for preparing heterocyclic methanone compounds comprising an amide coupling reaction of a heterocyclic carboxylic acid compound with an aza-bicyclo compound, in which one or both compounds can be provided in the form of salts as starting materials for the coupling reaction. The present disclosure also relates to compounds prepared by any processes as described herein and any compositions comprising the compounds.

Accordingly, in one aspect, there is provided a process for preparing a protected aza-bicyclo compound of Formula 4:

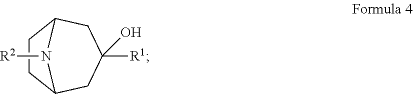

comprising a Grignard reaction of a nortropinone compound of Formula 5:

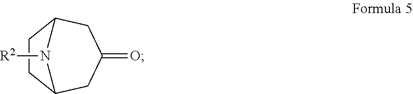

with a halogenated compound of Formula 6:

X—R¹ Formula 6;
wherein
R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³, and each R³ and R⁴ are independently selected from the group consisting of hydrogen and —C₁₋₆alkyl;
R² is an amine protecting group; and
X is a halogen.

In another aspect, there is provided a process for preparing a heterocyclic methanone compound of Formula 1:

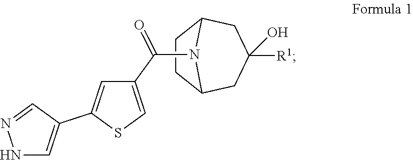

comprising reacting a carboxylic acid compound of Formula 2 or salt thereof:

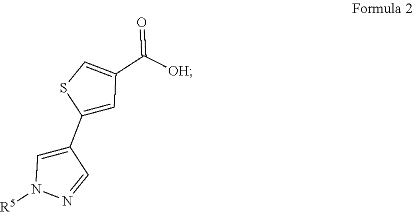

with an amine compound of Formula 3 or salt thereof, in the presence of at least one coupling reagent selected from an oxime coupling reagent and a carbodiimide coupling reagent:

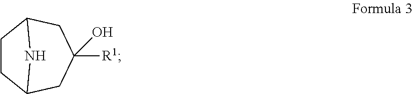

wherein
R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, C₁₋₆haloalkyl, —O—C₁₋₆haloalkyl, —CN, —NR³R⁴, —COR³, —CO₂R³, and each R³ and R⁴ are independently selected from the group consisting of hydrogen and C₁₋₆alkyl;

R⁵ is hydrogen or an amine protecting group.

In another aspect, there is provided a process for preparing a heterocyclic methanone compound of Formula 1:

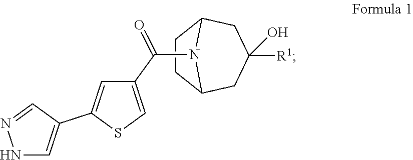

comprising reacting a carboxylic acid compound of Formula 2 or salt thereof:

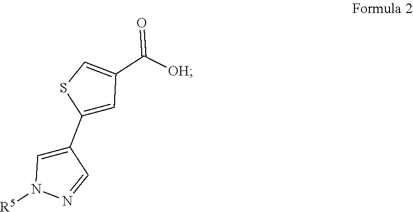

with a single or double salt of an amine compound of Formula 3, in the presence of at least one amide coupling reagent:

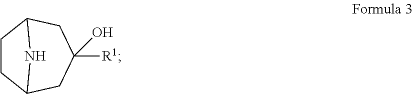

wherein
R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, C₁₋₆haloalkyl, —O—C₁₋₆haloalkyl, —CN, —NR³R⁴, —COR³, —CO₂R³, and each R³ and R⁴ are independently selected from the group consisting of hydrogen and C₁₋₆alkyl;
R⁵ is hydrogen or an amine protecting group.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE DRAWINGS

Whilst it will be appreciated that a variety of embodiments of the disclosure may be utilised, in the following, we describe a number of examples of the disclosure with reference to the following drawings.

FIG. 1 shows an HPLC chromatogram of crude Compound A8 reaction mixture at 1.5 h, following Grignard reaction with i-PrMgBr, Boc-nortropinone, and LaCl₃ in THF.

FIG. 2 shows an HPLC chromatogram of crude Compound A8 following Grignard reaction with i-PrMgBr, Boc-nortropinone, and LaCl₃ in THF.

FIG. 3 shows an HPLC chromatogram of purified Compound A8 following Grignard reaction with i-PrMgBr, Boc-nortropinone, and LaCl₃ in THF.

FIG. 4 shows an HPLC chromatogram of crude Compound A8 following scaled-up Grignard reaction with excess i-PrMgBr (1.7 eq.).

FIG. 5 shows a ¹H NMR spectrum of crude Compound A8 following scaled-up Grignard reaction with excess i-PrMgBr (1.7 eq.).

FIG. 6 shows an HPLC chromatogram of crude Compound A8 following scaled-up Grignard reaction with a deficit i-PrMgBr (1.3 eq.).

FIG. 7 shows a 1H NMR spectrum of crude Compound A8 following scaled-up Grignard reaction with a deficit i-PrMgBr (1.3 eq.).

FIG. 8 shows an HPLC chromatogram of the p-TSA salt of Compound A9 following scaled-up (30 g-50 g) telescoped reaction.

FIG. 9 shows a ¹H NMR spectrum of benzoic acid salt of Compound A9 following salt screening.

FIG. 10 shows a 1H NMR spectrum of p-TSA salt of Compound A9 following salt screening.

FIG. 11 shows a ¹H NMR spectrum of components of mixture from which the product Compound A9 was extracted, showing TsOH remaining.

FIG. 12 shows an HPLC chromatogram of purified Compound 1 following amide coupling reaction in p-TSA with Compound A9.

FIG. 13 shows an HPLC chromatogram of purified Compound 1 following amide coupling reaction with Oxymapure and EDC in THF.

FIG. 14 shows an HPLC chromatogram of purified Compound 1 following recrystallization from EtOH/H₂O 1:1.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

As used herein, the term about, unless stated to the contrary, refers to +/−20%, typically +/−10%, typically +/−5%, of the designated value.

As used herein, the terms “a”, “an” and “the” include both singular and plural aspects, unless the context clearly indicates otherwise.

The compounds of the present disclosure may contain chiral (asymmetric) centers or the molecule as a whole may be chiral. The individual stereoisomers (enantiomers and diastereoisomers) and mixtures of these are within the scope of the present invention.

As used herein, the term “halogen” means fluorine, chorine, bromine, or iodine.

As used herein, the term “alkyl” encompasses both straight chain (i.e., linear) and branched chain hydrocarbon groups. Examples of alkyl groups include methyl, ethyl, n-propyl, iso-propyl, n-butyl, t-butyl, i-butyl, sec-butyl, pentyl, and hexyl groups. In one example, the alkyl group is of one to six carbon atoms (i.e., C₁₋₆alkyl).

As used herein, the term “carbocyclyl” refers to an aromatic or non-aromatic cyclic group of carbon atoms. A carbocyclyl group may, for example, be monocyclic or polycyclic (i.e. bi-cyclic, tricyclic). A polycyclic carbocyclyl group may contain fused rings. In one example, the carbocyclyl group is of three to ten carbon atoms (i.e. C₃₋₁₀carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may be, for example, monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). Examples of monocyclic non-aromatic heterocyclyl groups include aziridinyl, azetidinyl, pyrrolidinyl, imidazolidinyl, py-razolidinyl, piperidinyl, piperazinyl, tetrahydrofuranyl, tetrahydropyranyl, morpholinyl, thi-omorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidine. Examples of bicyclic aromatic heterocyclyl groups (also referred to as bicyclic heteroaryl groups) include quinoxalinyl, quinazolinul, pyridopyrazinyl, benzoxazolyl, benzothiophenyl, ben-zimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

As used herein, the term “anion” refers to an ion bearing a negative charge. Similarly, as used herein, the term “cation” refers to an ion bearing a positive charge.

The present disclosure relates to compounds of Formula 1 and salts thereof. Salts may be formed in the case of embodiments of the compound of Formula 1, which contain a suitable acidic or basic group. Suitable salts of the compound of Formula 1 include those formed with organic or inorganic acids or bases. As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or tri-ethanolamine A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of Formula 1 and salts thereof.

Those skilled in the art of organic and/or medicinal chemistry will appreciate that the compounds of Formula 1 and salts thereof may be present in amorphous form, or in a crystalline form. It will be understood that the present disclosure encompasses all forms and polymorphs of the compounds of Formula 1 and salts thereof.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

### Process for Preparing Xanamem

The subject matter of the present disclosure is predicated in part on the surprising discovery of an efficient and scalable process for preparing Xanamem. Scheme 1, below, provides a non-limiting example of an efficient and scalable process for preparing Xanamem and related compounds (compounds of Formula 1).

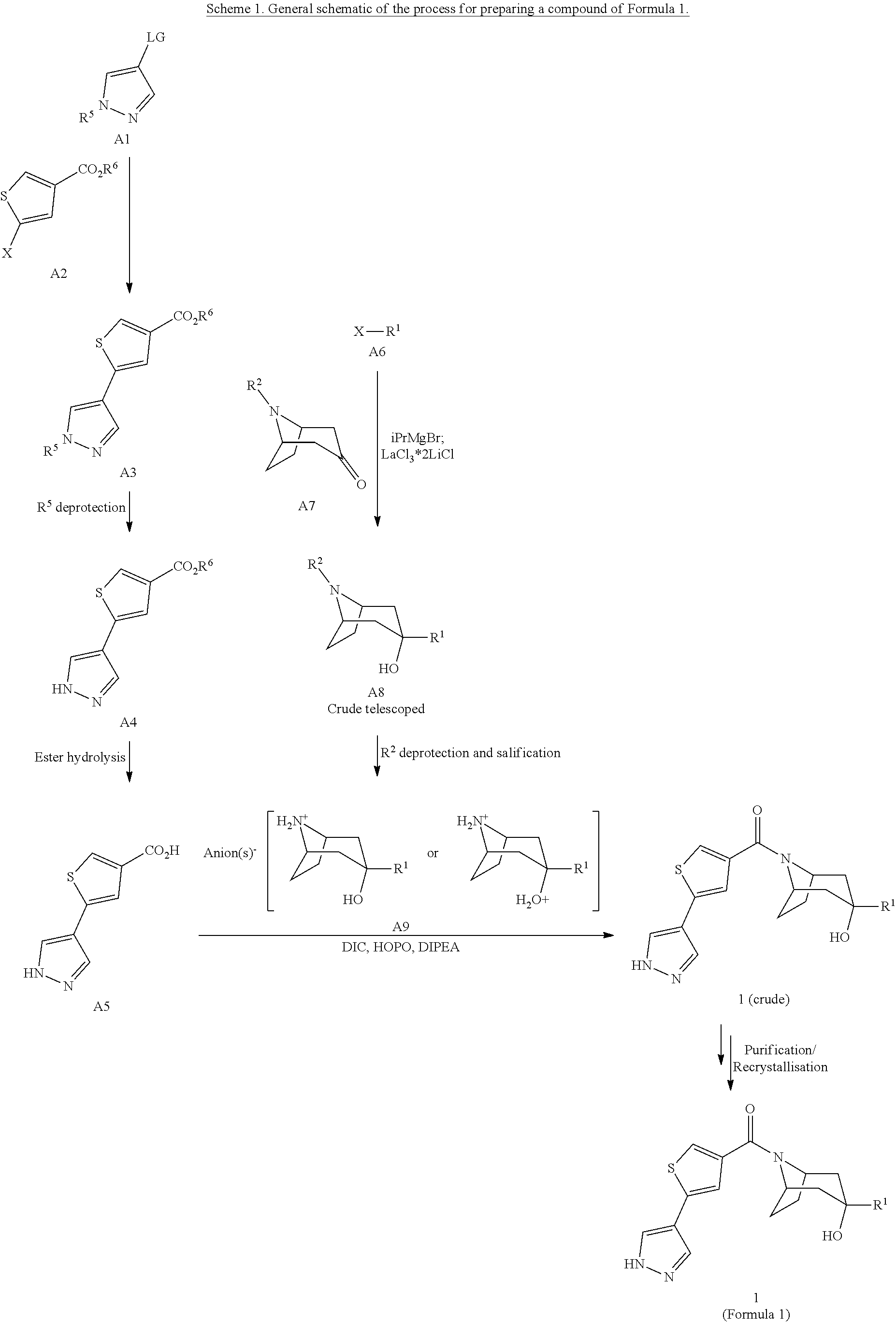

The above process is described further below in relation to each of the steps of the process. Each step may provide its own independent process aspect, embodiment or example for preparing an intermediate or compound per se, or may provide a further embodiment or example to another process aspect or embodiment as described herein. Each intermediate or prepared compound of each step may also provide its own independent aspect, embodiment or example, in relation to compounds, compositions and/or processes thereof.

### Synthesis of Compound A3

In some embodiments, Compound A3 is prepared by the reaction of Compound A1 with Compound A2.

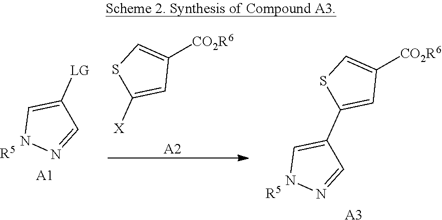

As used herein, the term “LG” refers to a “leaving group”, and may be any molecular fragment that departs with a pair of electrons in a heterolytic bond cleavage. In some embodiments, the leaving group (LG) is an anion. In some embodiments, the leaving group (LG) is a cation. In some embodiments, the leaving group (LG) is a neutral molecular fragment. Examples of anionic leaving groups (LG) include, but are not limited to, halides. In some embodiments, the leaving group (LG) is a halide. In some embodiments, the leaving group (LG) is a halide, and is selected from the group consisting of chlorine (Cl⁻), bromine (Br⁻), and iodine (I⁻). In one example, LG is chlorine (Cl⁻). In one example, LG is bromine (Br⁻). In one example, LG is iodine (I⁻). In some embodiments, LG is a boronic ester derivative. The introduction of a boronic ester derivative may be brought about through a Miyaura borylation reaction. In one example, LG is a boronic ester derivative having the structure:

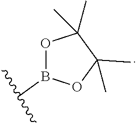 [SMILES: *B1OC(C)(C)C(C)(C)O1]

R⁵ may be a hydrogen or an amine protecting group. In some embodiments, R⁵ is a hydrogen. In some embodiments, R⁵ is an amine protecting group. As used herein, the term “protecting group” refers to a molecular fragment that chemically modifies a functional group to obtain chemoselectivity in a subsequent chemical reaction. The term “amine protecting group” specifically refers to a protecting group that chemically modifies an amine functional group to obtain chemoselectivity in a subsequent chemical reaction. Examples of amine protecting groups include, but are not limited to, carbamate, amide, benzyl, benzylidene, tosyl, and trityl protecting groups. In some embodiments, R⁵ is an amino protecting group selected from the group consisting of a carbamate, amide, benzyl, benzylidene, tosyl, and trityl protecting group. Examples of carbamate protecting groups include, but are not limited to, methyl and ethyl groups, 9-fluoroenylmethyl, 9-fluoroenylmethyloxycarbonyl (Fmoc), tert-butyloxycarbonyl (Boc), benzyl carbamate (Cbz), and p-methoxybenzyl carbonyl (MeOZ) groups. In some embodiments, R⁵ is a tert-butyloxycarbonyl (Boc) protecting group. Examples of amide protecting groups include, but are not limited to, acetyl (Ac), benzamide, trifluoroacetamide, trichloroacetamide, phenylacetamide, picolinamide, and phthalimide groups. Further examples of amino protecting groups include, but are not limited to, benzoyl, benzyl, benzylidene, p-methoxybenzyl (PMB), 3,4-dimethoxybenzyl (DMPM), p-methoxyphenyl (PMP), tosyl (Ts), trichloroethyl chloroformate (Troc), toluene sulphonyl, trityl, and triphenylmethyl groups.

In some embodiments, R⁵ is a tetrahydropyran (THP) moiety, being:

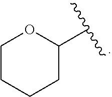 [SMILES: *C1CCCCO1]

In some embodiments, Compound A1 is:

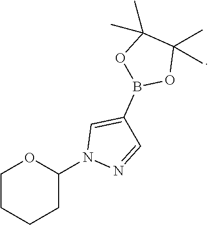 [SMILES: CC1(C)OB(c2cnn(C3CCCCO3)c2)OC1(C)C]

X may be a functional group capable of reacting with the leaving group (LG) of Compound A1, so as to form a carbon-carbon single bond. In some embodiments, X is a halide. In some embodiments, X is selected from the group consisting of chlorine, bromine, and iodine. In one example, X is chlorine. In one example, X is bromine. In one example, X is iodine.

R⁶ may be hydrogen or an ester protecting group. In some embodiments, R⁶ is a hydrogen. In some embodiments, R⁶ is an ester protecting group. As used herein, the term “ester protecting group” refers to a molecular fragment that chemically modifies an ester functional group to obtain chemoselectivity in a subsequent chemical reaction. In some embodiments, R⁶ is a linear or branched alkyl chain. In some embodiments, R⁶ is a linear or branched C₁₋₆alkyl chain. In some embodiments, R⁶ is a C₁₋₆alkylaryl group. In some embodiments, R⁶ is selected from the group consisting of methyl (CH₃), ethyl (CH₂CH₃), propyl (CH₂ CH₂ CH₃), benzyl, and t-butyl (C(CH₃)₃). In one example, R⁶ is a methyl group. In one example, R⁶ is an ethyl group. In one example, R⁶ is a benzyl group.

In some embodiments, Compound A2 is:

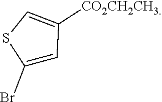

Compound A1 is reacted with Compound A2 to form Compound A3, under suitable conditions as understood by the person skilled in the art. Various carbon-carbon bond forming reaction conditions are known in the art. In some embodiments, Compound A1 is reacted with Compound A2 under Suzuki reaction conditions to afford Compound A3. Suzuki reaction conditions may also be referred to as Suzuki-Miyaura reaction conditions, or as a Suzuki coupling. As will be understood by the person skilled in the art, a Suzuki reaction is a cross-coupling reaction in which the coupling partners are a boronic acid/ester derivative and an organohalide, whereby the reaction is catalysed by a metal catalyst in the presence of a base.

The metal catalyst is typically a palladium catalyst, though may also be a nickel catalyst. In some embodiments, the reaction is catalysed by a palladium catalyst. In some embodiments, the reaction is catalysed by a nickel catalyst. In some embodiments, the reaction is catalysed by a catalyst selected from the group consisting of Pd(Amphos)₂Cl₂, Pd(PPh₃)₄, Pd₂(dba)₃, Pd(OAc)₂, PdCl₂ (dppf), Ni(cod)₂, NiCl₂-glyme, NiCl₂(PCy₃)₂, NiCl₂(dppp), and NiCl₂(PPh₃)₂. In one example, the metal catalyst is Pd(Amphos)₂Cl₂. In some embodiments, relative to Compound A2, between about 0.01 to 0.1 equivalents, between about 0.01 to 0.05 equivalents, or between about 0.02 to 0.025 equivalents of metal catalyst is employed in the reaction.

The reaction may be further catalysed by a phosphine ligand derivative. Examples of such ligands include, but are not limited to, BrettPhos, AdBrettPhos, tBuBrettPhos, RuPhos, CPhos, AlPhos, SPhos, XPhos, MePhos, JohnPhos, CyJohnPhos, XantPhos, and DavePhos.

The base is typically a water-soluble base. In some embodiments, the base is selected from the group consisting of potassium carbonate (K₂CO₃), potassium t-butoxide (KOtBu), caesium carbonate (Cs₂CO₃), tripotassium phosphate (K₃PO₄), sodium hydroxide (NaOH), and triethyl amine (NEt₃). In one example, the base is potassium carbonate (K₂CO₃). In some embodiments, relative to Compound A2, between about 1 to 5 equivalents, between about 1 to 2 equivalents, or between about 1 to 1.5 equivalents of base is employed in the reaction.

The reaction may be conducted in a variety of suitable solvent systems, as would be understood by the person skilled in the art. In some embodiments, the solvent is an aqueous solvent, such as a mixture comprising water. In some embodiments, the solvent is a biphasic mixture comprising water. In some embodiments, the solvent is a biphasic mixture comprising water and one or more ether solvents. The aqueous solvent or biphasic mixture may comprise or consist of solvents selected from water, a polar ether solvent, a non-polar ether solvent, or combinations thereof. Further advantages were unexpectedly provided by the use of biphasic mixtures, such as further reducing any minor impurities, for example catalyst such as palladium.

In some embodiments, the reaction is performed in a polar solvent, such as a polar protic solvent, polar aprotic solvent, or combination thereof. In some embodiments, the reaction is performed in a non-polar solvent, such as a non-polar aprotic solvent. Examples of polar protic solvents include, but are not limited to, water, alcohols and glycols. Examples of alcohols include, but are not limited to, methanol (MeOH), ethanol (EtOH), 1-propanol, isopropyl alcohol (2-propanol, iPrOH or IPA), 1-butanol, 2-butanol, t-butanol (t-BuOH), 1-pentanol, 3-methyl-1-butanol, and 2-methyl-1-propanol. Examples of glycols include, but are not limited to, ethylene glycol. Examples of polar aprotic solvents include, but are not limited to, halogenated hydrocarbons, ketones, nitriles, esters, carbonate esters, ethers, sulfoxides, sulfones, amides, nitroalkanes, and pyrrolidines. Examples of ketones include, but are not limited to, acetone, methylethyl ketone (MEK), methylbutyl ketone (MBK), methylisobutyl ketone (MIBK), and methylisopropyl ketone. Examples of nitriles include, but are not limited to, acetonitrile (MeCN). Examples of esters include, but are not limited to, ethyl formate, methyl acetate (MeOAc), ethyl acetate (EtOAc), propyl acetate, isopropyl acetate (iPAC), n-butyl acetate, and isobutyl acetate. Examples of carbonate esters include, but are not limited to, dimethyl carbonate (DMC) and propylene carbonate (PC). Examples of polar and non-polar ethers include, but are not limited to, methyl-tert-butyl ether (MTBE), diethyl ether, 1,4-dioxane, 2-methoxyethanol, 2-ethoxyethanol, dimethoxyethane (DME or monoglyme), 1,1-dimethoxymethane, 2,2-dimethoxypropane, 1,1-diethoxypropane, isopropyl ether, petroleum ether, cyclopentyl methyl ether (CPME), anisole (methoxybenzene), methyltetrahydrofuran (MeTHF), and tetrahydrofuran (THF). Examples of sulfoxides include, but are not limited to, dimethylsulfoxide (DMSO). Examples of sulfones include, but are not limited to, sulfolane. Examples of amides include, but are not limited to, formamide, N,N-dimethylacetamide, and N,N-dimethylformamide (DMF). Examples of nitroalkanes include, but are not limited to, nitromethane. Examples of pyrrolidines include, but are not limited to, N-methylpyrrolidone (NMP). Examples of polar and non-polar halogenated hydrocarbons, such as chlorocarbons, include, but are not limited to, dichloromethane (DCM), chloroform, 1,2-dichloroethane, 1,1,1-trichloroethane, 1,1-dichloroethene, and 1,2-dichloroethene. In one example, the reaction is performed in an ether, such as CPME and MeTHF.

In some embodiments, the solvent comprises or consists of water and one or more polar aprotic ether solvents, such as CPME and MeTHF.

In one example, the reaction conditions employ Pd(Amphos)₂Cl₂ as the catalyst, potassium carbonate (K₂CO₃) as the base, and ether/water as the solvent. The ether may be a polar ether according to any examples as described herein, such as CPME and/or MeTHF.

Compound A3 is formed by the reaction, in which R⁵ and R⁶ are as described herein. In one example, Compound A3 is:

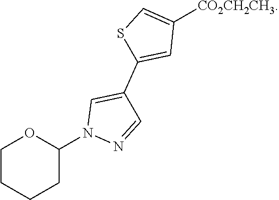

In the synthesis of Xanamem and its analogues, Compound A3 may be utilised in the sequential synthetic steps either without purification (i.e., obtained and reacted as the crude reaction product) or may be firstly isolated and/or purified. Suitable isolation and/or purification techniques would be appreciated by the person skilled in the art.

### Synthesis of Compound A4

In some embodiments, Compound A4 is prepared by the deprotection of R⁵ from Compound A3.

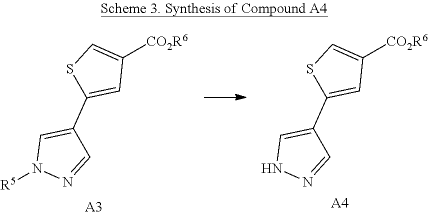

R⁵ and R⁶ are as described herein. Compound A3 is reacted under suitable reaction conditions to form Compound A4, as would be understood by the person skilled in the art. The deprotection of R⁵ results in the free secondary amine (—N(H)—) of Compound A4.

In some embodiments, R⁵ is:

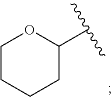 [SMILES: *C1CCCCO1]

and acidic reaction conditions are required to deprotect the amine to which R⁵ is attached. In one example, the acidic reaction conditions include hydrochloric acid (HCl). An excess of hydrochloric acid may be required. In some embodiments, at least about 1.5, 2, 3, 4, or 5 equivalents of hydrochloric acid (HCl) relative to Compound A3 is employed in the reaction. In one example, about 4 equivalents of hydrochloric acid (HCl) relative to Compound A3 is employed in the reaction.

The person skilled in the art will appreciate that a variety of suitable solvents may be employed for the reaction. Any one or more of the above solvents previously described for the preparation of a Compound A3 may be provided for the reaction in preparing a Compound A4. In one example, the solvent is a biphasic solvent according to any examples as described herein. In one example, the solvent comprises an ester and/or an ether. In another example, the solvent comprises an ether, such as cyclopentyl methyl ether (CPME) and 2-methyltatrahydrofuran (2-MeTHF).

The person skilled in the art will appreciate that it may be necessary to apply heat to facilitate the reaction. In some embodiments, the reaction is heated to between about 30° C. and 80° C., about 40° C. and 70° C., or about 45° C. and 55° C. In one example, the reaction is heated to about 50° C.

Purification may be provided by recrystallization, which in some example may be achieved using solvents selected from an ester and/or ether.

### Synthesis of Compound A5

In some embodiments, Compound A5 is prepared by the hydrolysis of R⁶ from Compound A4.

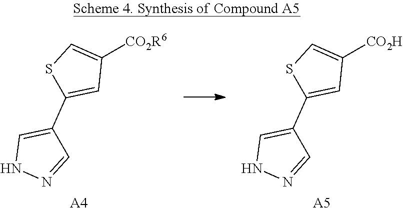

R⁶ is as described herein. Compound A4 is reacted under suitable reaction conditions so as to hydrolyse R⁶ to afford Compound A5, as would be understood by the person skilled in the art. In some embodiments, the reaction is an ester hydrolysis reaction. Hydrolysis of the R⁶ group affords the carboxylic acid group on Compound A5.

The hydrolysis reaction may be acid- or base-catalysed. In some embodiments, the hydrolysis reaction is acid-catalysed. In some embodiments, the hydrolysis reaction is base-catalysed. Examples of suitable acids include, but are not limited to, hydrochloric acid (HCl). Examples of suitable bases include, but are not limited to, sodium hydroxide (NaOH), potassium hydroxide (KOH), and lithium hydroxide (LiOH). In some embodiments, the hydrolysis reaction is base catalysed by lithium hydroxide (LiOH). In one example, the base is in the form of lithium hydroxide monohydrate (LiOH·H₂O).

The person skilled in the art will appreciate that a variety of suitable solvents may be employed for the reaction. Any one or more of the above solvents previously described for the preparation of a Compound A3 or Compound A4 may be provided for the reaction in preparing a Compound A5. In one example, the solvent is a biphasic solvent according to any examples as described herein. In one example, the solvent comprises an ester and/or an ether. In another example, the solvent comprises an ether, such as cyclopentyl methyl ether (CPME) and 2-methyltatrahydrofuran (2-MeTHF).

The person skilled in the art will appreciate that it may be necessary to apply heat to facilitate the reaction. In some embodiments, the reaction is heated to between about 30° C. and 70° C., about 30° C. and 50° C., or about 30° C. and 40° C. In one example, the reaction is heated to about 35° C.

### Synthesis of Compound A8

In some embodiments, Compound A8 is prepared by a reaction of Compound A6 with Compound A7.

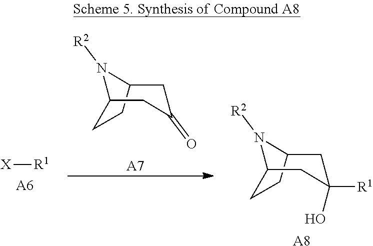

In some embodiments, there is provided a process for preparing a protected amine Compound A8 of Formula 4:

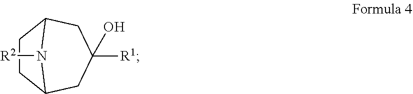

comprising a Grignard reaction of a nortropinone Compound A7 of Formula 5:

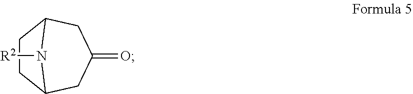

with a halogenated Compound A6 of Formula 6:

X—R¹ Formula 6.

In some embodiments, R¹ is a carbocyclyl or heterocyclyl. In one example, R¹ is a carbocyclyl. In one example, R¹ is a heterocyclyl. In some embodiments, each carbocyclyl or heterocyclyl is a monocyclic or bicyclic group. In one example, the carbocyclyl is a monocyclic group. In one example, the carbocyclyl is a bicyclic group. In one example, the heterocyclyl is a monocyclic group. In one example, the heterocyclyl is a bicyclic group. In some embodiments, each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³. In some embodiments, each carbocyclyl and heterocyclyl is a monocyclic or bicyclic group each unsubstituted. In some embodiments, each carbocyclyl and heterocyclyl is a monocyclic or bicyclic group each substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³.

In some embodiments, R¹ is a monocyclic or bicyclic heteroaryl group each unsubstituted or substituted with one or more substituents selected from the group consisting of halogen, —OH, C₁₋₆haloalkyl, O—C₁₋₆halo alkyl. In some embodiments, R¹ is pyrimidine unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl. In some embodiments, R¹ is an unsubstituted pyrimidine.

In some embodiments, R² is an amine protecting group as described herein. In one example, R² is an amine protecting group selected from the group consisting of carbamate (e.g. tert-butyloxycarbonyl (BOC), t-butyl carbamate BOC, 9-fluorenylmethyl carbamate FMOC, benzyl carbamate CBZ), amide (e.g. acetamide Ac, trifluoroacetamide, phthalimide), benzyl, benzylidene, tosyl (e.g. toluene sulphonyl), and trityl (e.g. triphenylmethyl). In one example, R² is a tert-butyloxycarbonyl (BOC) group.

In some embodiments, R³ and R⁴ are independently selected from the group consisting of hydrogen and C₁₋₆alkyl. In one example, R³ is hydrogen. In one example, R³ is C₁₋₆alkyl. In one example, R⁴ is hydrogen. In one example, R⁴ is C₁₋₆alkyl.

In some embodiments, X is a halogen. In some embodiments, X is selected from the group consisting of chlorine, bromine, and iodine. In some embodiments, X is chlorine. In some embodiments, X is bromine. In some embodiments, X is iodine.

In some embodiments, the Grignard reaction comprises the steps of i) a halogen-metal exchange reaction including a Grignard reagent and ii) a coupling reaction including LaCl₃.

In some embodiments, the Grignard reagent is selected from the group consisting of i-PrMgBr, i-PrMgCl·LiCl (“Turbo Grignard” reagent), and sec-BuMgCl·LiCl. In one example, the Grignard reagent is i-PrMgBr.

In some embodiments, the halogen-metal exchange reaction advantageously obviates the need for cryogenic cooling conditions. In some embodiments, the halogen-metal exchange reaction including i-PrMgBr is undertaken between about −40° C. and 20° C., about −30° C. and 10° C., or about −20° C. and 0° C. In one example, the halogen-metal exchange reaction including i-PrMgBr is undertaken between about −20° C. and 0° C. In one example, the halogen-metal exchange reaction including i-PrMgBr is undertaken between about −20° C. and −15° C. In some embodiments, the i-PrMgBr is added to the reaction mixture between about −20° C. and −15° C.

In some embodiments, the halogen-metal exchange reaction including i-PrMgBr is undertaken using between about 1 and 3 equivalents of i-PrMgBr, about 1 and 2 equivalents of i-PrMgBr, or about 1.1 and 1.5 equivalents of i-PrMgBr. In some examples, the halogen-metal exchange reaction including i-PrMgBr is undertaken using equivalents of i-PrMgBr in at least about 1, 1.1, 1.2, 1.3, 1.4, or 1.5. In some examples, the halogen-metal exchange reaction including i-PrMgBr is undertaken using equivalents of i-PrMgBr in less than about 3, 2.5. 2. 1.9, 1.8, 1.7, 1.6, 1.5, 1.4, 1.3, or 1.2. In some examples, the halogen-metal exchange reaction including i-PrMgBr is undertaken using equivalents of i-PrMgBr in an amount between any two of the previous upper and/or lower amounts.

In some embodiments, following the complete addition of i-PrMgBr, the reaction mixture is stirred for t minutes before the mixture is allowed to warm to about 0° C. In some embodiments, t is between about 5 and about 60 minutes, about 10 and 45 minutes, or about 20 and 40 minutes. In some embodiments, t is about 5 minutes, about 10 minutes, about 20 minutes, about 30 minutes, about 45 minutes, or about 60 minutes. In one example, t is about 30 minutes. That is, in one example, the duration of the halogen-metal exchange reaction is about 30 minutes.

Once the reaction mixture is allowed to warm to about 0° C., the coupling reaction including LaCl₃ is undertaken. In some embodiments, the coupling reaction occurs at about 0° C., about 1° C., about 2° C., about 3° C., about 4° C., or about 5° C. In some embodiments, the coupling reaction occurs between about 0° C. and 20° C., about 0° C. and 10° C., or about 0° C. and 5° C. In one example, the coupling reaction occurs between about 0° C. and 5° C.

In some embodiments, the amount of LaCl₃ utilised in the reaction is between about 1 and 3 equivalents, about 1.1 and 2 equivalents, about 1.2 and 1.8 equivalents, or about 1.4 and 1.6 equivalents. In one example, the amount of LaCl₃ utilised in the reaction is about 1.5 equivalents.

In one example, the LaCl₃ is LaCl₃·2LiCl.

The person skilled in the art will appreciate that a variety of suitable solvents may be employed for the reaction. Any one or more of the above solvents previously described for the preparation of a Compound A3, Compound A4, or Compound A5, may be provided for the reaction in preparing a Compound A8. In one example, the solvent comprises an ester and/or an ether. In another example, the solvent comprises an ether, such as cyclopentyl methyl ether (CPME) and 2-methyltatrahydrofuran (2-MeTHF). The solvent may be present in the reaction in any amount suitable so as to effect the reaction. In some examples the solvent may be anhydrous. For example, the amount of water in the solvent may be less than about (in ppm) 500, 400, 300, 200, 100, 75, 50, 25, 10, 5, or 1.

In some embodiments, the reaction may be monitored for quenching of the Grignard reaction prior to complete conversion to the Compound A8, such as when an amount of Grignard reagent is added such that conversion is at least 50%, 75%, 90%, or 95%. The reaction mixture may be quenched by pouring onto an acid such as an aqueous solution comprising citric acid.

In some embodiments, the Compound A8 of Formula 4 is a compound of Formula 4a:

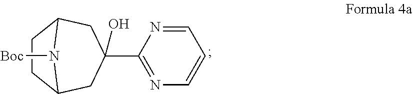 [SMILES: CC(C)(C)OC(=O)N1C2CCC1CC(O)(c1ncccn1)C2]

and the process comprises reacting a Compound A7 of Formula 5a:

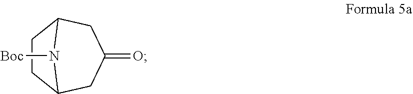 [SMILES: CC(C)(C)OC(=O)N1C2CCC1CC(=O)C2]

with a Compound A6 of Formula 6a:

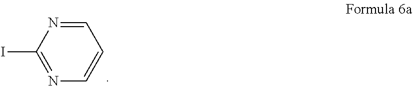

In the synthesis of Xanamem and its analogues, a Compound A8 of Formula 4 may or may not be purified prior to being progressed through subsequent synthetic steps or reactions. In one example, a Compound A8 of Formula 4 is purified. Conventional purification through column chromatography is suitable for isolating a Compound A8 of Formula 4 in good purity. In one example, a Compound A8 is purified by column chromatography. In one example, a Compound A8 is not purified prior to being progressed through subsequent synthetic reactions. That is, the crude material is directly reacted in the synthesis of a Compound A9 of Formula 3. Such carry through of the crude material is referred to in the art as “telescoping” the crude material into the subsequent chemical reaction.

### Synthesis of Compound A9

In some embodiments, Compound A9 is prepared by deprotecting R² from Compound A8, and optionally forming a salt of Compound A9. The salt may be formed as a single or double salt, for example as follows:

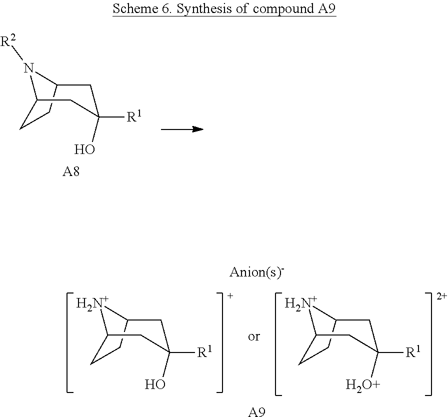

R¹ and R² can be provided according to any embodiments or examples thereof as described herein.

In some embodiments, there is provided a process for preparing an aza-bicyclo Compound A9 of Formula 3, or a salt thereof:

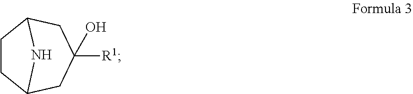

wherein the process comprises removing an amine protecting group from a Compound A8 of Formula 4, and optionally salification thereof.

The Compound of A8 may be used as a crude product from its previous reaction as described, for example directly telescoped to be a starting material in preparing a Compound of A9 of Formula 3.

It will be understood that the amine protecting group may be removed by any suitable methods known in the art, depending upon the nature of the protecting group. In some embodiments, the amine protecting group is removed under acidic conditions, such as with acids including hydrochloric, acetic, or sulphonic. In one example, the protecting group is a BOC protecting group, and it is removed under acidic conditions. In one example, the protecting group is a BOC protecting group, and it is removed under aqueous hydrochloric acid (HCl) conditions. In one example, the protecting group is a BOC protecting group, and it is removed under trifluoroacetic acid (TFA) conditions. In one example, the acidic conditions comprise sulphonic acid. The sulphonic acid may be an optionally substituted alkyl or aromatic sulphonic acid, such as p-toluenesuphonic acid (also known as tosylic acid TsOH). In one example, the protecting group is a BOC protecting group, and it is removed under sulphonic acid conditions, such as with tosylic acid (TsOH). Further advantages may be provided using sulphonic acids, such as fast precipitation of the reaction product to form single or double tosylate salt.

The Compound A9 of Formula 3 may optionally be subject to salification. As used herein, the term “salification” refers to the conversion of a chemical to its salt form. In some embodiments, a Compound A9 of Formula 3 is reacted in subsequent chemical reactions in its salt form. Conversion of Compound A9 of Formula 3 to its salt may result in a more stable intermediate (e.g., less susceptible to degradation). The person skilled in the art will appreciate that numerous suitable salts may be utilised. In one example, the salification is prepared using a sulphonic acid, such as para-toluenesulfonic acid (p-TSA or TsOH), to form a tosylate salt of a Compound A9 of Formula 3. Sulphonic acid has been found effective to provide a dual function of deprotection of the amine protecting group and salification of the resulting deprotected compound. The salification can provide a single or double salt, such as a bis-tosylate salt (e.g. pTSA:nortropinone in a range of 1:1 to 2:1). The salt formation has been found to provide further advantages in purification with fast crystallisation and precipitation from solutions to provide a stable salt compound that may be used directly (e.g. without further purification) in subsequent amide coupling reaction, particularly when prepared as double salt.

The person skilled in the art will appreciate that a variety of suitable solvents may be employed for the reaction. Any one or more of the above solvents previously described for the preparation of a Compound A3, Compound A4, Compound A5, or Compound A8, may be provided for the reaction in preparing a Compound A9. In one example, the solvent is selected from the group consisting of water, alcohol, ester, ether, or combination thereof. The solvent may be an aqueous solvent. The solvent may comprise an acid according to any examples as previously described above. In one example, the solvent comprises an ether, such as cyclopentyl methyl ether (CPME) and 2-methyltatrahydrofuran (2-MeTHF). In another example, solvent comprises an alcohol, such as isopropyl alcohol (IPA). The solvent may be present in the reaction in any amount suitable so as to effect the reaction. The acid may be present in the reaction in an amount (in mol/L) of between about 0.1 and 2, 0.2 and 1, or 0.3 and 0.7. The acid may be present in the reaction in an amount in molar equivalents of a Compound A9 of Formula 3 in an amount of at least 1, 1.5, 2.0, 2.5, 3.0, 3.5, 4.0, 4.5, or 5, and/or in an amount of less than about 5.5, 5.0, 4.5, 4.0, 3.5, 3.0, 2.5, 2.0, or 1.5, or in a range amount provided by any two of these upper and/or lower values such as between 2 and 5 or between 2.5 and 4.5.

### Synthesis of Compound 1

In some embodiments, a heterocylic methanone Compound 1 is prepared by an amide coupling reaction between a carboxylic acid Compound A5 or salt thereof, and an aza-bicyclo Compound A9 or salt thereof.

In some embodiments, there is provided a process for preparing a heterocylic methanone Compound 1 of Formula 1:

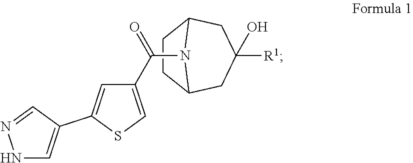

wherein R¹ is any embodiment or example thereof as defined herein;
comprising reacting a carboxylic acid compound of Formula 2 or a salt thereof:

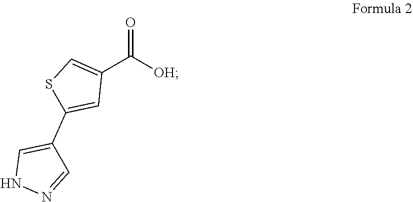 [SMILES: O=C(O)c1csc(-c2cn[nH]c2)c1]

with an aza-bicyclo compound of Formula 3 or salt thereof as prepared herein:

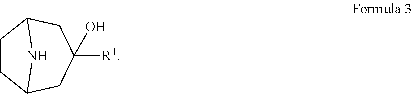

In some embodiments, the carboxylic acid Compound A5 is provided as a salt, such as a halide salt (e.g. chloride).

In some embodiments, the aza-bicyclo Compound A9 is provided as a salt, such as a single salt, double salt, or combination thereof, as follows:

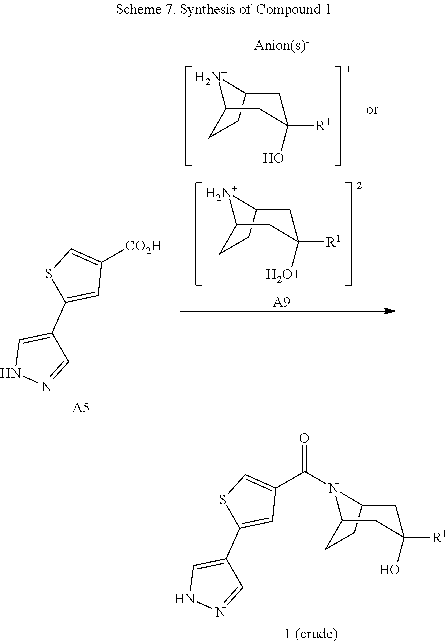

In some examples, the single or double salt of the aza-bicyclo Compound A9 is a sulphonate salt, such as a tosylate salt according to any examples thereof as described herein.

In some examples, the Compound A9 of Formula 3 is a double sulphonate salt of Formula 3a:

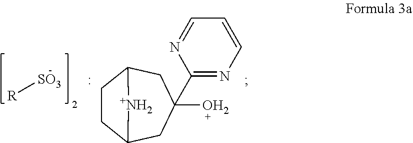

wherein R is selected from an alkyl, aryl and alkyl aryl, each of which are optionally substituted.

Examples of sulphonate salts include mesylate (methanesulfonate), triflates (trifluoromethanesulfonate), ethanesulfonate (esylates), tosylate (p-toluenesulfonate), benzenesulfonate (besylate), closilate (closylate, chlorobenzenesulfonate), camphorsulfonate (camsylate), pipsylate (p-iodobenzenesulfonate), or nosylate. In one example, the sulphonate salt is a tosylate.

In some embodiments, an equimolar or an excess molar equivalent of the carboxylic acid Compound A5 or salt thereof, is used with respect to the aza-bicyclo Compound A9 or salt thereof. For example, the molar equivalents of carboxylic acid Compound A5 or salt thereof with respect to the aza-bicyclo Compound A9 or salt thereof is at least 1, 1.05, 1.1, 1.15, 1.2, 1.25, 1.3, 1.35, 1.4, 1.45, or 1.5.

In some embodiments, there is provided a process for preparing a heterocyclic methanone compound of Formula 1:

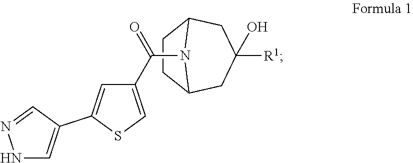

comprising reacting a carboxylic acid compound of Formula 2 or a salt thereof:

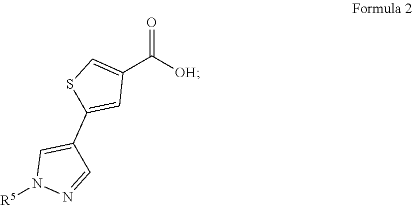

with an amine compound of Formula 3 or salt thereof, in the presence of at least one coupling reagent selected from an oxime coupling reagent and a carbodiimide coupling reagent:

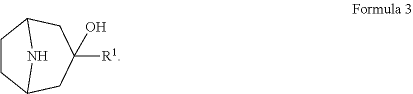

R¹ in Formula 3 can be selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³, and each R³ and R⁴ are independently selected from the group consisting of hydrogen and C₁₋₆alkyl. R⁵ can be hydrogen or an amine protecting group, according to any embodiments or examples thereof as described herein.

In some embodiments, R¹ is a carbocyclyl or heterocyclyl. In some embodiments, each carbocyclyl or heterocyclyl is a monocyclic or bicyclic group. In one example, the carbocyclyl is a monocyclic group. In one example, the carbocyclyl is a bicyclic group. In one example, the heterocyclyl is a monocyclic group. In one example, the heterocyclyl is a bicyclic group. In some embodiments, each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³. In some embodiments, each carbocyclyl and heterocyclyl is a monocyclic or bicyclic group each unsubstituted. In some embodiments, each carbocyclyl and heterocyclyl is a monocyclic or bicyclic group each substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³.

In some embodiments, R¹ is a monocyclic or bicyclic heteroaryl group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl. In some embodiments, R¹ is pyrimidine unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl. In some embodiments, R¹ is an unsubstituted pyrimidine.

As used herein, the term “coupling reagent” refers to a compound that can create a chemical bond between two chemical moieties. In one example, the coupling reagent is an “amide coupling reagent”, and provides a chemical bond between a carboxylic acid moiety and an amine moiety, thereby forming an amide bond. The coupling reagent may be optionally accompanied by the use of one or more additives or one or more base compounds for facilitating the coupling reaction.

In some embodiments, the amide coupling reagent is at least one coupling reagent selected from the group consisting of a carbodiimide coupling reagent and an oxime coupling reagent. In some embodiments, the amide coupling reagent is a carbodiimide coupling reagent. In some embodiments, the carbodiimide coupling reagent is selected from the group consisting of DCC (dicyclohexylcarbodiimide), DIC (diisopropylcarbodiimide), EDAC·HCl (N-(3-dimethylaminopropyl)-N′-ethylcarbodiimide·HCl), EDC (1-ethyl-3-(3-dimethylaminopropyl) carbodiimide), and combinations thereof. In one example, the carbodiimide coupling reagent is EDC (1-ethyl-3-(3-dimethylaminopropyl) carbodiimide). In one example, the carbodiimide coupling reagent is DIC (diisopropylcarbodiimide).

In some embodiments, the coupling reagent is an oxime coupling reagent. In some embodiments, the oxime coupling reagent is selected from the group consisting of OxymaPure (2-cyano-2-(hydroxyimino)acetate), K-Oxyma (potassium 2-cyano-2-(hydroxyimino)-acetate), COMU (1-[(1-(cyano-2-ethoxy-2-oxoethylideneaminooxy)dimethyl-aminomorph-olinomethylene)]methanaminium hexafluorophosphate), PyOxym-M, PyOxim (O-[(cyano(ethoxycarbonyl)-methyliden)amino]yloxytripyrrolidinophosphonium hexafluorophosphate), HONM (isonitroso Meldrum's acid), Ocyma-B, Oxyma-T, Amox, HMMU, Fmoc-Amox, and combinations thereof. In one example, the oxime coupling reagent is OxymaPure (2-cyano-2-(hydroxyimino)acetate).

In some embodiments, at least 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, or 3.5, equivalents of the amide coupling reagent, relative to Compound A5, is used in the reaction. In some embodiments, less than 5, 4.5, 4, 3.5, 3, 2.9, 2.8, 2.7, 2.6, 2.5, 2.4, 2.3, 2.2, 2.1, 2, 1.9, 1.8, 1.6, or 1.5, equivalents of the amide coupling reagent, relative to Compound A5 or Formula 2, is used in the reaction. The equivalents of the amide coupling reagent, relative to Compound A5 or Formula 2, used in the reaction may be provided in a range between any two of these upper and/or lower values, for example between about 1 and 3, 1.2 and 2, or 1.3 and 1.7. It will be appreciated that in one example the amide coupling reagent is a carbodiimide coupling reagent (e.g. DIC), and the process optionally further comprises one or more additives (e.g. HOPO and/or DIPEA), according to any examples thereof as described herein.

An additive may be used with the amide coupling reagent. An additive may be any reagent that facilitates/catalyses the amide coupling reaction. In one example, the additive is an N-oxide reagent such as 2-hydroxypyridine-N-oxide (HOPO). It will be appreciated that the N-oxide reagent has an N⁺—O⁻ bond, for example an optionally substituted pyridine N-oxide such as HOPO. For example, the reagents may comprise or consist of a carbodiimide coupling reagent and an optional additive.

In some embodiments, at least 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, or 3.5, equivalents of the additive (e.g. HOPO), relative to Compound A5, is used in the reaction. In some embodiments, less than 5, 4.5, 4, 3.5, 3, 2.9, 2.8, 2.7, 2.6, 2.5, 2.4, 2.3, 2.2, 2.1, 2, 1.9, 1.8, 1.6, or 1.5, equivalents of the additive, relative to Compound A5, is used in the reaction. The equivalents of the additive, relative to Compound A5, used in the reaction may be provided in a range between any two of these upper and/or lower values, for example between about 1 and 4, 1.1 and 3, or 1.2 and 2.

In some embodiments, the base is present in an amount of less than about 10, 9, 8, 7, 6, 5, 4, 3, 2, 1, 0.5, or 0.1 equivalents, relative to the molar amount of the compound of Compound A5. In some embodiments, the base is present in an amount of greater than about 0.1, 0.3, 0.5, 0.7, 1, 1.5, 2, 2.5, 3, or 3.5 equivalents, relative to the molar amount of Compound A5. In some embodiments, the base is present in a range provided by any two of the above upper and/or lower amounts of the additive, such as between 1 and 7, 2 and 6, or 2.5 and 4.5.

In another example, the reagents may comprise or consist of a carbodiimide coupling reagent and optionally one or more additives. In one example, the additive is an N-oxide reagent, such as 2-hydroxypyridine-N-oxide (HOPO). In one example, the additive is a base, such as an amine (e.g. DIPEA). In one example, the reagents comprise or consist of a carbodiimide coupling reagent (e.g. diisopropylcarbodiimide), an N-oxide additive (e.g. 2-hydroxypyridine-N-oxide), and a base additive (e.g. DIPEA).

In some embodiments, the coupling reagent is selected from at least one oxime coupling reagent and at least one carbodiimide coupling reagent, which may each be provided according to any embodiments or examples thereof as described herein. In one example, the coupling reagent is selected from the group consisting of OxymaPure (2-cyano-2-(hydroxyimino) acetate), EDC (1-ethyl-3-(3-dimethylaminopropyl) carbodiimide) and 2-hydroxypyridine-N-oxide (HOPO). It will be appreciated that one or more optional additives may also be used according to any examples thereof as described herein.

It has been surprisingly found that the use of at least one carbodiimide coupling reagent can enable the amide coupling reaction to occur without any significant formation of a any undesirable by-products (e.g. tetramethylurea, TMU). In some embodiments, the process involves the use of the specific combination of at least one carbodiimide coupling reagent, optionally at least one additive (e.g. N-oxide such as HOPO), and optionally at least one base (e.g. DIPEA), wherein the presence of undesirable by-products is substantially reduced or circumvented (e.g. tetramethylurea, TMU). In some embodiments, the process involves the use of DIC, optionally with HOPO and/or DIPEA.

The person skilled in the art will appreciate that a variety of suitable solvents may be employed for the reaction. Any one or more of the above solvents previously described for the preparation of a Compound A3, Compound A4, Compound A5, Compound A8, or Compound A9, may be provided for the reaction in preparing a compound of Formula 1. In one example, the solvent is selected from the group consisting of water, alcohol, ester, ether, nitrile, or combination thereof. The solvent may be an aqueous solvent. In one example, the solvent comprises an ether, such as cyclopentyl methyl ether (CPME) and 2-methyltatrahydrofuran (2-MeTHF). In another example, solvent comprises an alcohol, such as isopropyl alcohol (IPA). In another example, the solvent comprises a nitrile, such as acetonitrile. In another example, the solvent comprises acetonitrile. The solvent may be present in the reaction in any amount suitable so as to effect the reaction. In one example, the solvent in an aqueous solvent comprising water and one or more organic solvents according to any examples as described herein (e.g. a nitrile solvent such as acetonitrile).

In some embodiments, the reaction comprises an organic solvent that is a polar protic or aprotic solvent. Examples of polar, aprotic solvents include, but are not limited to, acetonitrile (ACN), dimethylformamide (DMF), dichloromethane (DCM), tetrahydrofuran (THF), ethyl acetate (EtOAc), dimethyl sulfoxide (DMSO), acetone, hexamethylphosphoric triamide (HMPT), dimethyl ketone, and methylethyl ketone. In one example, the organic solvent is a polar, aprotic solvent being acetonitrile (ACN). In one example, the organic solvent is Me-THF.

In some embodiments, the reaction is provided in an aqueous solvent, for example water and a water miscible solvent such as acetonitrile. Examples of suitable water miscible solvents include alcohols, ethers, and nitriles. In one example, the aqueous solvent is a mixture of water and acetonitrile, such as in a ratio of about 1:3 to about 3:1, or about 1:1.

In some embodiments, further solvents are added to the reaction mixture following substantial completion of the reaction to facilitate precipitation of a compound of Formula 1, such as an alcohol (e.g. ethanol).

In some examples, the reaction mixture, comprising Compound A5, the carbodiimide coupling reagent, and additives selected from HOPO and DIPEA, are stirred for about 10 minutes, about 20 minutes, about 30 minutes, about 1 hour, or about 6 hours, prior to the addition of Compound A9. In some embodiments, the reaction mixture is heated to a temperature between about 30 and 90° C., 40 to 80° C., or 50 to 70° C. Examples of solvents include aqueous solvents such as water and acetonitrile (e.g. about 1:1).

In some embodiments, the Compound A9 of Formula 3 is a secondary amine salt according to any examples as described herein. In one example, the Compound A9 of Formula 3 is a secondary amine sulphonate salt, such as a para-toluenesulfonic acid (p-TSA) salt.

In some embodiments, there is provided a process wherein the compound of Formula 1 is a compound of Formula 1a:

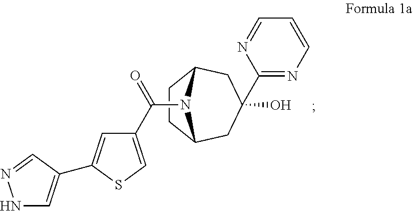 [SMILES: O=C(c1csc(-c2cn[nH]c2)c1)N1[C@@H]2CC[C@H]1C[C@](O)(c1ncccn1)C2]

comprising reacting a carboxylic acid compound of Formula 2a or salt thereof:

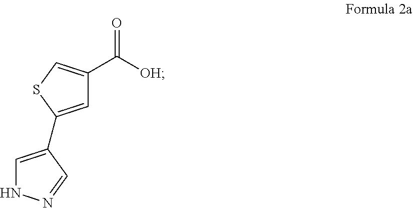 [SMILES: O=C(O)c1csc(-c2cn[nH]c2)c1]

with a sulphonte (e.g p-TSA) salt compound of Formula 3a in the presence of a carbodiimide coupling reagent and optionally one or more additives:

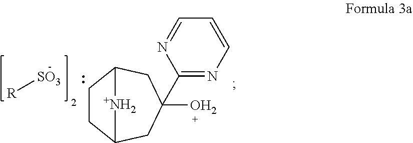

wherein R is selected from an alkyl, aryl and alkyl aryl, each of which are optionally substituted.

In some embodiments, the carboxylic acid compound of Formula 2 is prepared by saponification of an ester compound of Formula 7:

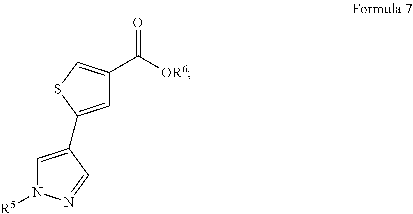

with a base, wherein R⁵ is hydrogen or an amine protecting group and R⁶ is an ester protecting group, as described herein.

It will be appreciated that R⁶ may be cleaved from the compound of Formula 7 by base-catalysed hydrolysis. In some embodiments, the base is selected from the group consisting of sodium hydroxide (NaOH), lithium hydroxide (LiOH), and potassium hydroxide (KOH). In one example, the base is lithium hydroxide (LiOH). Alternatively, it will be appreciated that R⁶ may be cleaved from the compound of Formula 7 by acid catalysed hydrolysis.

In some embodiments, the R⁵ amine protecting group in the compound of Formula 7 is removed prior to preparing the carboxylic acid compound of Formula 2.

### Scale-Up

The process as described herein, allows for the scalable synthetic pathway and manufacture of a compound of Formula 1. The process as described, when compared to the process described in international patent application WO2011135276, provides increased overall yield of Compound 1, scalable reaction conditions, and obviates the production of potentially toxic by-products.

In some embodiments, the process is conducted on small-scale (e.g., scale of 20 mg to 1 gram), as would be suitable for research and development purposes. However, in some other embodiments, the process is conducted on large-scale (e.g., scale of greater than 1 gram, particularly greater than 50 grams), as would be suitable for manufacturing purposes. The synthesis or one or more steps thereof may occur as a batch-type process.

In some embodiments, the process for preparing of a compound of Formula 4 occurs with a starting material amount of a compound of Formula 5 or a compound of Formula 6 of at least 1 g, at least 10 g, at least 50 g, at least 100 g, at least 500 g, at least 1 kg, or at least 10 kg. That is, the process for preparing a compound of Formula 4 occurs on at least 1 g, at least 10 g, at least 50 g, at least 100 g, at least 500 g, at least 1 kg, or at least 10 kg scale. In one example, the process for preparing Compound A8 occurs with a starting material amount of Compound A7 or Compound A6 of at least 1 g, at least 10 g, at least 50 g, at least 100 g, at least 500 g, at least 1 kg, or at least 10 kg. That is, the process for preparing Compound A8 occurs on at least 1 g, at least 10 g, at least 50 g, at least 100 g, at least 500 g, at least 1 kg, or at least 10 kg scale.

In some embodiments, the process provides a conversion of a compound of Formula 5 to a compound of Formula 4 of at least 40%, at least 50%, at least 60%, at least 70%, at least 75%, or at least 80%, as measured by HPLC. It will be understood that the conversion of a reaction may be measured at any point during the reaction, through any suitable technique, such as TLC or HPLC. Typically, an aliquot of the reaction mixture will be subject to HPLC, where the relevant component peaks are identified and integrated relative to one another. In some embodiments, the Grignard reaction, as described herein, provides a conversion of a compound of Formula 5 to a compound of Formula 4 of at least 40%, at least 50%, at least 60%, at least 70%, at least 75%, or at least 80%, as measured by HPLC.

As used herein, the term “yield” will be taken to mean the amount of either crude or purified compound obtained from a reaction, measured as a percentage of theoretical yield of the compound in that reaction, as would be understood by the person skilled in the art.

In some embodiments, the process provides a yield of a compound of Formula 4 of at least 20%, at least 30%, at least 40%, at least 50%, at least 60%, at least 70%, or at least 80%, as determined from a compound of Formula 5 and a compound of Formula 6 starting materials. That is, in some embodiments, the Grignard reaction, as described herein, provides at least 20%, at least 30%, at least 40%, at least 50%, at least 60%, at least 70%, or at least 80% yield of a compound of Formula 4. In some embodiments, the Grignard reaction, as described herein, provides between about 20% and 80%, between about 30% and 70%, or between about 50% and 70% yield of a compound of Formula 4. In one example, the Grignard reaction, as described herein, provides at least 20%, at least 30%, at least 40%, at least 50%, at least 60%, at least 70%, or at least 80% yield of Compound A8. In some embodiments, the Grignard reaction, as described herein, provides between about 20% and 80%, between about 30% and 70%, or between about 50% and 70% yield of Compound A8.

In some embodiments, the process described herein provides a compound of Formula 4 in high purity. As would be understood by a skilled person, purity is a measure independent of yield. That is, a compound may have a high purity, albeit a low yield. As used herein, the term “high purity” refers to at least 80% of the ultimately obtained material being the desired compound (e.g., Formula 4), which may be measured, for example, by HPLC methods. The purity of a compound may be measured based on the crude reaction mixture, the product isolated from the reaction mixture (i.e., following the reaction work-up), or the purified product (i.e., following chromatography, recrystallization, etc.).

In some embodiments, the Grignard reaction, as described herein, provides a compound of Formula 4 in at least 30%, 40%, 50%, 60%, 70%, 80%, 85%, 90%, or 95% purity. In one example, the Grignard reaction, as described herein, provides a compound of Formula 4 in at least 30%, 40%, or 50% purity of the product in the crude reaction mixture. In one example, the Grignard reaction, a described herein, provides a compound of Formula 4 in at least 50% purity of the product isolated from the reaction mixture (i.e., following the reaction work-up). In one example, the Grignard reaction, as described herein, provides a compound of Formula 4 in at least 95% purity following purification. In one example, the Grignard reaction, as described herein, provides a compound of Formula 4 in at least 95% purity following recrystallization. In one example, the Grignard reaction, as described herein, provides a compound of Formula 4 in at least 95% purity following column chromatography.

In some embodiments, there is provided a process for preparing an aza-bicyclic compound of Formula 4:

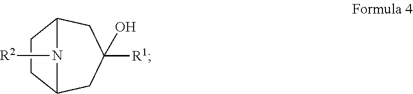

comprising a Grignard reaction of a nortropinone compound of Formula 5:

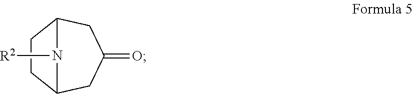

with a halogenated compound of Formula 6:

X—R¹ Formula 6;
wherein R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³, and each R³ and R⁴ are independently selected from the group consisting of hydrogen and —C₁₋₆alkyl; R² is an amine protecting group; and X is a halogen; and wherein the yield of a compound of Formula 4 is at least 20%, at least 30%, at least 40%, at least 50%, at least 60%, at least 70%, or at least 80%.

In some embodiments, the process for preparing of a compound of Formula 1 occurs with a starting material amount of a compound of Formula 2 or a compound of Formula 3 of at least 1 g, at least 10 g, at least 50 g, at least 100 g, at least 500 g, at least 1 kg, or at least 10 kg. That is, the process for preparing a compound of Formula 1 occurs on at least 1 g, at least 10 g, at least 50 g, at least 100 g, at least 500 g, at least 1 kg, or at least 10 kg scale. In one example, the process for preparing Compound 1 occurs with a starting material amount of Compound A5 or Compound A9 of at least 1 g, at least 10 g, at least 50 g, at least 100 g, at least 500 g, at least 1 kg, or at least 10 kg. That is, the process for preparing Compound 1 occurs on at least 1 g, at least 10 g, at least 50 g, at least 100 g, at least 500 g, at least 1 kg, or at least 10 kg scale.

In some embodiments, the process provides a conversion of a compound of Formula 2 to a compound of Formula 1 of at least 40%, at least 50%, at least 60%, at least 70%, at least 75%, or at least 80%, as measured by HPLC. In some embodiments, the process provides a conversion of a compound of Formula 3 to a compound of Formula 1 of at least 40%, at least 50%, at least 60%, at least 70%, at least 75%, or at least 80%, as measured by HPLC. In some embodiments, the amide coupling reaction, as described herein, provides a conversion of a compound of Formula 2 to a compound of Formula 1 of at least 40%, at least 50%, at least 60%, at least 70%, at least 75%, or at least 80%, as measured by HPLC. In some embodiments, the amide coupling reaction, as described herein, provides a conversion of a compound of Formula 3 to a compound of Formula 1 of at least 40%, at least 50%, at least 60%, at least 70%, at least 75%, or at least 80%, as measured by HPLC.

In some embodiments, the process provides a yield of a compound of Formula 1 of at least 20%, at least 30%, at least 40%, at least 50%, at least 60%, at least 70%, or at least 80%, as determined from a compound of Formula 2 and a compound of Formula 3 starting materials. That is, in some embodiments, the amide coupling reaction, as described herein, provides at least 20%, at least 30%, at least 40%, at least 50%, at least 60%, at least 70%, or at least 80% yield of a compound of Formula 1. In some embodiments, the amide coupling reaction, as described herein, provides between about 20% and 80%, between about 30% and 70%, or between about 50% and 70% yield of a compound of Formula 1. In one example, the amide coupling reaction, as described herein, provides at least 20%, at least 30%, at least 40%, at least 50%, at least 60%, at least 70%, or at least 80% yield of Compound 1. In some embodiments, the amide coupling reaction, as described herein, provides between about 20% and 80%, between about 30% and 70%, or between about 50% and 70% yield of Compound 1.

In some embodiments, the process described herein provides a compound of Formula 1 in high purity. In some embodiments, the amide coupling reaction, as described herein, provides a compound of Formula 1 in at least 80%, at least 85%, at least 90%, at least 95%, at least 96%, at least 97%, at least 98%, or at least 99% purity. In one example, the amide coupling reaction, as described herein, provides a compound of Formula 1 in at least 80% purity of the product in the crude reaction mixture. In one example, the amide coupling reaction, as described herein, provides a compound of Formula 1 in at least 80% purity of the product isolated from the reaction mixture (i.e., following the reaction work-up). In one example, the amide coupling reaction, as described herein, provides a compound of Formula 1 in at least 95% purity following purification. In one example, the amide coupling reaction, as described herein, provides a compound of Formula 1 in at least 95% purity following recrystallization. In one example, the amide coupling reaction, as described herein, provides a compound of Formula 1 in at least 95% purity following column chromatography.

In some embodiments, there is provided a process for preparing a heterocyclic methanone compound of Formula 1:

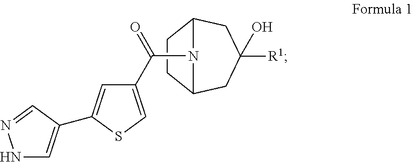

comprising reacting a carboxylic acid compound of Formula 2 or a salt thereof:

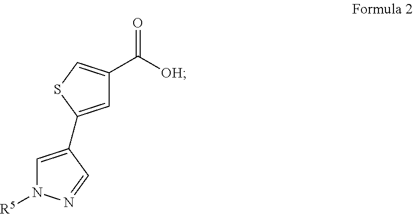

with an amine bicyclic compound of Formula 3 or a salt thereof, in the presence of at least one coupling reagent:

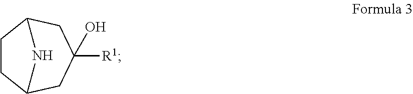

wherein R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, C₁₋₆haloalkyl, —O—C₁₋₆haloalkyl, —CN, —NR³R⁴, —COR³, —CO₂R³, and each R³ and R⁴ are independently selected from the group consisting of hydrogen and C₁₋₆alkyl; R⁵ is hydrogen or an amine protecting group; wherein the yield of a compound of Formula 1 is at least 20%, at least 30%, at least 40%, at least 50%, at least 60%, at least 70%, or at least 80%.

### Compounds

In some embodiments, there is provided a compound of Formula 1:

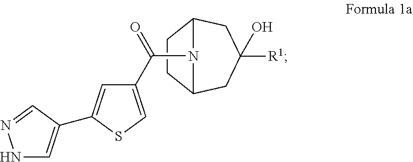

wherein R¹ is as described herein, prepared by any process as described herein.

In some embodiments, there is provided a compound of Formula 1a:

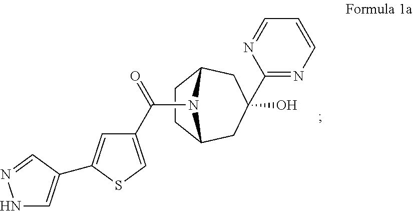 [SMILES: O=C(c1csc(-c2cn[nH]c2)c1)N1[C@@H]2CC[C@H]1C[C@](O)(c1ncccn1)C2]

prepared by any process as described herein.

In some embodiments, there is provided a compound of Formula 4:

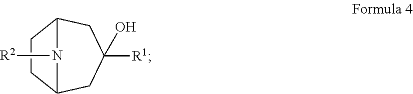

wherein R¹ is as described herein, prepared by any process as described herein.

In some embodiments, there is provided a compound of Formula 4a:

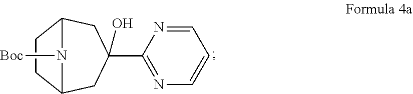 [SMILES: CC(C)(C)OC(=O)N1C2CCC1CC(O)(c1ncccn1)C2]

prepared by any process as described herein.

In some embodiments or examples there may be provided one or more of the intermediate compounds as described herein in any of the steps of the process.

### Compositions

Whilst a compound of Formula 1 or salt thereof may in some embodiments be administered alone, it is more typically administered as part of a pharmaceutical composition or formulation. Thus, the present disclosure also provides a pharmaceutical composition comprising a compound of Formula 1 or salt thereof and a pharmaceutically acceptable excipient. The pharmaceutical composition comprises one or more pharmaceutically acceptable diluents, carriers or excipients (collectively referred to herein as “excipient” materials).

The present disclosure also provides pharmaceutical formulations or compositions, both for veterinary and for human medical use, which comprise compounds of Formula 1 of the present disclosure or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof.

Examples of pharmaceutical formulations include those suitable for oral, parenteral (including subcutaneous, intradermal, intramuscular, intravenous, and intraarticular), inhalation (including fine particle dusts or mists that may be generated by means of various types of metered dose pressurised aerosols), nebulisers or insufflators, rectal, intraperitoneal and topical (including dermal, buccal, sublingual, and intraocular) administration, although the most suitable route may depend upon, for example, the condition and disorder of the recipient.

The pharmaceutical formulations may conveniently be presented in unit dosage form and may be prepared by any of the methods well known in the art of pharmacy. All methods include the step of bringing a compound of Formula (I) or salt thereof into association with the excipient that constitutes one or more necessary ingredients. In general, the formulations are prepared by uniformly and intimately bringing into association the active ingredient with liquid carriers or finely divided solid carriers or both, and then, if necessary, shaping the product into the desired formulation.

In some embodiments, that composition is formulated for oral delivery. For example, pharmaceutical formulations of the present invention suitable for oral administration may be presented as discrete units such as capsules, cachets, pills or tablets each containing a predetermined amount of the active ingredient; as a powder or granules, as a solution or a suspension in an aqueous liquid or non-aqueous liquid, for example as elixirs, tinctures, suspensions or syrups; or as an oil-in-water liquid emulsion or a water-in-oil liquid emulsion. A compound of Formula 1 may also be presented as a bolus, electuary or paste.

A tablet may be made for example by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Moulded tablets may be made by moulding in a suitable machine a mixture of the powdered compound moistened with an inert liquid diluent. The tablets may be optionally coated or scored, and may be formulated so as to provide slow or controlled release of the compound of Formula 1. The compound of Formula 1 can, for example, be administered in a form suitable for immediate release or extended release Immediate release or extended release can be achieved by the use of suitable pharmaceutical compositions comprising a compound of Formula 1 or, particularly in the case of extended release, by the use of devices such as subcutaneous implants or osmotic pumps. A compound of Formula 1 may also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavouring agents such as those well known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents, and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Disintegrators include without limitation, starch, methylcellulose, agar, bentonite, xanthan gum, and the like. A compound of Formula 1 can also be delivered through the oral cavity by sublingual and/or buccal administration. Moulded tablets, compressed tablets, or freeze-dried tablets are exemplary forms that may be used. Exemplary compositions include those formulating a compound of Formula 1 with fast dissolving diluents such as mannitol, lactose, sucrose and/or cyclodextrins. Also included in such formulations may be high molecular weight excipients such as cellulose (avicel) or polyethylene glycols (PEGs). Such formulations can also include an excipient to aid mucosal adhesion such as hydroxyl propyl cellulose (HPC), hydroxyl propyl methyl cellulose (HPMC), sodium carboxy methyl cellulose (SCMC), maleic anhydride copolymer, and agents to control release such as polyacrylic copolymer. Lubricants, glidants, flavours, colouring agents, and stabilisers may also be added for ease of fabrication and use. Lubricants used in these dosage forms include sodium oleate, sodium stearate, magnesium stearate, sodium benzoate, sodium acetate, sodium chloride, and the like. For oral administration in liquid form, the oral drug components can be combined with any oral, non-toxic, pharmaceutically acceptable inert carrier such as ethanol, glycerol, water, and the like.

In some embodiments, the composition is formulated for parenteral delivery. Formulations for parenteral administration include aqueous and non-aqueous sterile injections solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1.3-butanediol, water, Ringer's solution, an isotonic sodium chloride solution, or other suitable dispersing or wetting and suspending agents, including synthetic mono- or diglycerides, and fatty acids, including oleic acid, or Cremaphor.

For example, in one embodiment, the formulation may be a sterile, lyophilized composition that is suitable for reconstitution in an aqueous vehicle prior to injection. In one embodiment, a formulation suitable for parenteral administration conveniently comprises a sterile aqueous preparation of the compound of Formula 1, which may for example be formulated to be isotonic with the blood of the recipient.

The compounds of Formula 1 of the present disclosure may for example be formulated in compositions including those suitable for inhalation to the lung, by aerosol, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the compound of Formula 1 into association with a carrier that constitutes one or more accessory ingredients. In general, the compositions are prepared by bringing the compound of Formula 1 into association with a liquid carrier to form a solution or a suspension, or alternatively, bring the compound of Formula 1 into association with formulation components suitable for forming a solid, optionally a particulate product, and then, if warranted, shaping the product into a desired delivery form. Solid formulations of the present disclosure, when particulate, will typically comprise particles with sizes ranging from about 1 nanometer to about 500 microns. In general, for solid formulations intended for intravenous administration, particles will typically range from about 1 nm to about 10 microns in diameter. The composition may contain compounds of Formula 1 of the present disclosure that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In one example, the composition contains compounds of Formula 1 with a mean size of between 5 and 20 nm. In some embodiments, the compound of Formula 1 is polydispersed in the composition, with PDI of between 1.01 and 1.8, especially between 1.01 and 1.5, and more especially between 1.01 and 1.2. In one example, the compounds of Formula 1 are monodispersed in the composition.

It should be understood that in addition to the ingredients particularly mentioned above, the formulations may include other agents conventional in the art having regard to the type of formulation in question, for example, those suitable for oral administration may include flavouring agents.

The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

In some embodiments, there is provided a compound of Formula 1a in a purity of at least about 90, 91, 92, 93, 94, 95, 96, 97, 98, 99, 99.5, or 99.9 (weight % based on total composition comprising the compound of Formula 1a):

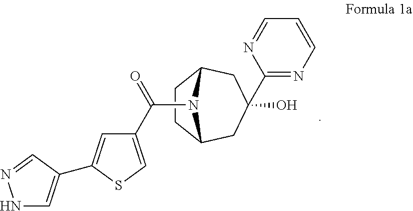 [SMILES: O=C(c1csc(-c2cn[nH]c2)c1)N1[C@@H]2CC[C@H]1C[C@](O)(c1ncccn1)C2]

For the compound of Formula 1a, the high purity may wherein if any impurities are present, then they are in an amount (weight % of the total weight of the composition) of less than about 5, 4, 3, 2, 1, 0.5, 0.1, 0.05, 0.01, 0.05, 0.001, 0.005, or 0.0001. The compound may be substantially free of any impurities. The impurities may be selected from any one or more of the by-products or reagents used in the processes as described herein, for example TMU, THP, and/or iodo pyrimidine. In one example the impurity, if present, is TMU. The high purity compound may be obtained from a crude reaction composition of the amide coupling reaction step in preparing the compound of Formula 1a. The compound may be a purified (e.g. washed and/or solvent extract) from the crude reaction composition. The high purity compound of Formula 1a may be provided in a pharmaceutical composition comprising one or more pharmaceutically acceptable excipients according to any embodiments or examples thereof as described herein.

In some embodiments, there is provided a composition comprising a compound of Formula 1a and one or more excipients according to any embodiments or examples thereof as described herein:

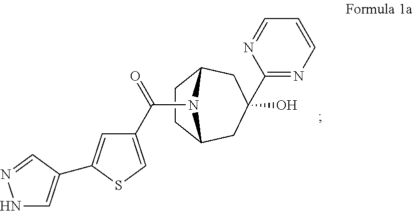 [SMILES: O=C(c1csc(-c2cn[nH]c2)c1)N1[C@@H]2CC[C@H]1C[C@](O)(c1ncccn1)C2]

wherein any impurities, if present, are in an amount (weight % of the total weight of the composition) of less than about 5, 4, 3, 2, 1, 0.5, 0.1, 0.05, 0.01, 0.005, 0.001, 0.0005 or 0.0001. The composition may be substantially free of any impurities. The impurities may be selected from any one or more of the by-products or reagents used in the processes as described herein, for example TMU, THP, DIPU, and/or iodo pyrimidine. In one example the impurity, if present, is TMU. The composition may be a crude reaction composition of the amide coupling reaction step in preparing the compound of Formula 1a. The composition may be a purified (e.g. washed and/or solvent extract) of the crude reaction composition. The composition may be a pharmaceutical composition comprising one or more pharmaceutically acceptable excipients according to any embodiments or examples thereof as described herein.

The present disclosure will now be described with reference to the following examples which illustrate some particular aspects of the present disclosure. However, it is to be understood that the particularity of the following description of the present disclosure is not to supersede the generality of the preceding description of the present disclosure.

### Examples

### General: Materials and Methods

Unless otherwise stated, all solvents and reagents were obtained from commercial sources.

TABLE 1
Abbreviations.

| | |
|---|---|
| API | Active pharmaceutical ingredient |
| Aq. | aqueous |
| Boc | tert-butyloxycarbonyl protecting group |
| Brine | Saturated aqueous sodium chloride solution |
| BRP | Batch record production |
| nBuLi | n-butyllithium |
| CPME | Cyclopentyl methyl ether |
| Eq. | equivalents |
| GC | Gas chromatography |
| HATU | Hexafluorophosphate Azabenzotriazole Tetramethyl Uronium |
| HCl | hydrochloric acid |
| HFR | High Force Research |
| HPLC | High Performance Liquid Chromatography |
| IT | Internal temperature |
| JT | Jacket temperature |
| MeCH | Methylcyclohexane |
| 2-MeTHF | 2-methyl tetrahydrofuran |
| MPLC | Medium pressure liquid chromatography |
| NAC | N-Acetyl-L-cysteine |
| NaOH | sodium hydroxide |
| qNMR | quantitative Nuclear Magnetic Resonance |
| RT | room temperature |
| THF | Tetrahydrofuran |
| TLC | Thin layer chromatography |
| TFA | Trifluoroacetic acid |
| THP | Tetrahydropyran |
| TMU | Tetramethylurea |

### Example 1: Synthesis of Compound A3

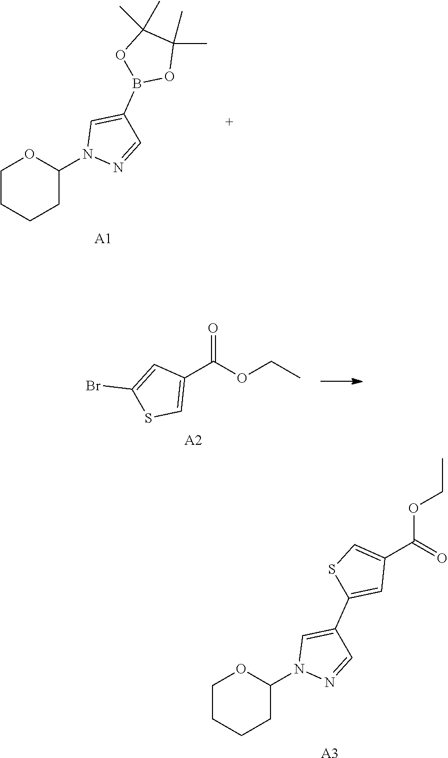

A1 (43.3 g) and bis(di-tert-butyl(4-dimethylaminophenyl)phosphine)dichloro-palladium (II) (933 mg) were charged in a reactor. Dioxane (582 mL), 5-bromothiophene-3-carboxylic acid ethyl ester (31 g) and a solution of K₂CO₃ (42.8 g) in water (95.9 mL) were added. The reaction mixture was heated to 85° C. IPC after 4 h showed full conversion (HPLC showed no residual A1, A3: 89 area %) and the reaction mixture was cooled to 25° C. (IT). Brine (110 mL) was added, the mixture was clear filtered, the phases were separated and the organic phase was evaporated under reduced pressure. The aqueous phase was extracted with 2-MeTHF (31 mL). The dioxane phase was evaporated under reduced pressure and 2-MeTHF (167 mL) was added and combined with the 2-MeTHF extraction phase. The combined organic phases were washed with NaHCO₃ (115 mL) and brine (110 mL). The product solution was stored at 2-8° C. for the following step. For yield determination, an aliquot was taken, evaporated and analyzed. The calculated crude A3 yield was determined to be 58.9 g (146%). The NMR assay corrected yield was 93%. The purity was determined as 85.5 area %.

### Example 2: Synthesis of Compound A4

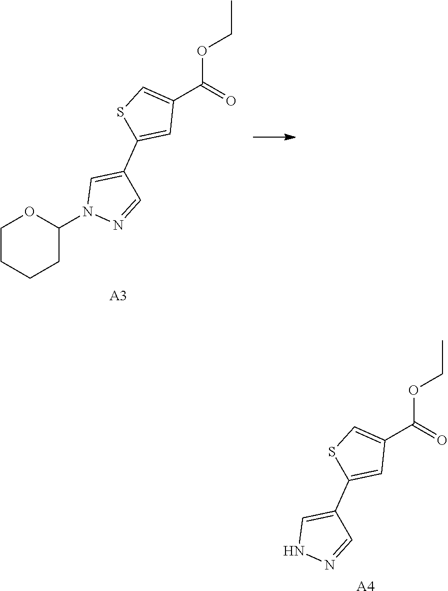

A reaction vessel was charged with the 2-methyltetrahydrofuran solution containing A3. Solution is heated up to 50° C. and 4 eq of hydrochloric acid are added slowly. Suspension was cooled down to 0° C. after the addition and stirred cold for 30 min. Suspension was then filtered and the solids are dried under reduced pressure. Light brown solids were suspended in 1 vol.eq of 2-methyltetrahydrofuran and a potassium carbonate solution is added dropwise until pH 10-12 is reached. Layers were separated from the biphasic solution. Solvents from the organic layer were removed under reduced pressure. Brown solid was suspended in 5 vol. eq. of isopropyl acetate and heated up to reflux and clear filtered. Clear solution was gradually cooled down to 0° C. and allowed to stir over night. Light brown suspension was filtered and solids dried to afford A4.

### Cleavage of THP Protection Group

Screening was undertaken to determine which acid, in what amount and at what temperature, was preferred for cleavage of the tetrahydropyran (THP) group. The results showed that an excess of hydrochloric acid, in water or isopropanol, at about 55° C. for approximately 24 hours, achieved good cleavage of the THP group. H₂SO₄ and ethanol, at about 80° C. for approximately 48 hours, also achieved good cleavage of the THP group, however, some saponification of the ester was also observed. Ultimately, hydrochloric acid was preferred as the reagent to cleave the THP group. Furthermore, it was decided to use an organic hydrochloric acid solution so that the A4 salt did not go into solution and can be cleanly filtered off and to prevent an equilibrium developing.

### Recrystallisation

Crystallisations were tested on a small scale in isopropanol and isopropyl acetate under the following conditions. Two vials were charged with 48 mg of A4 and diluted in 0.73 mL (15 vol. eq.) of isopropanol and isopropyl acetate respectively. Both were heated up to reflux (both became clear brown solutions) and then let to gradually cool down to room temperature. Both were filtered at room temperature, analysed by HPLC and yields determined.

| Experiment | Yield % | HPLC area % |
|---|---|---|
| IPA | 38.5% | 96.10% A4 |
| | | 1.26% of a new impurity (the isopropyl derivative |
| | | of A4) |
| iPrOAc | 52.7% | 96.57% A4 |

It was decided to perform the recrystallization in isopropyl acetate, due to better yields and no new impurities developing.

The remainder of the crude A4 from was diluted in 10 vol. eq. of isopropyl acetate and heated up to reflux. Substance dissolved roughly 10° C. before reflux. Solution cooled down gradually to 20° C. and solids were then filtered and dried at 50° C. under reduced pressure. Yield of the recrystallization on the larger scale was 77%.

The recrystallization was further optimized by using already purified material. A4 dissolves in 5 vol. eq. of isopropyl acetate at reflux. Mixture was then by clear filtered and allowed to cool. Solid was obtained with purity >98%.

### Example 3: Synthesis of Compound A5

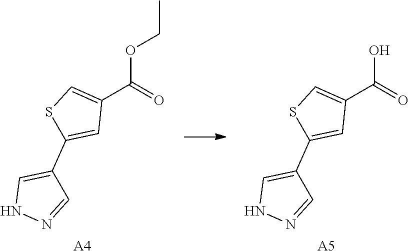

### Saponification Using Lithium Hydroxide Monohydrate

A solution of A4 in 2-methyltertrahydrofuran (2-Me-THF) was charged in a reaction vessel alongside a solution of lithium hydroxide monohydrate (3 eq.) in water (5 vol. eq.). The mixture was stirred at 35° C. overnight. Full conversion to A5 was observed. No work up or purification was performed.

### Saponification Using Sodium Hydroxide Solution

Solubility test showed that A5 was soluble in in water at pH 4. A saponification of A4 in aqueous conditions was conducted.

A4 was suspended in water (7.3 vol. eq.) and an aqueous sodium hydroxide solution, consisting of 1.3 eq of sodium hydroxide dissolved in 3 vol. eq. of water, was added. Mixture was then heated up to 65° C. Full conversion was observed after one hour. Mixture was cooled down to 45° C. and HCl was added dropwise until pH 5. Resulting suspension was cooled down to 10° C. and filtered. Solids were dried and analyzed by HPLC (97.86%). Yield: 86.91%.

### Example 4: Synthesis of Compound A8

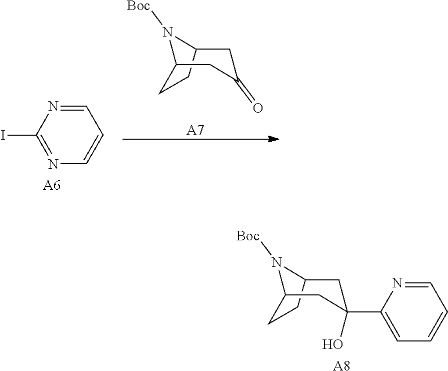

### Halogen-Metal Exchange Reaction

A screen of reagents for the halogen-metal exchange reaction was undertaken including the following Grignard reagents:
i-PrMg·LiCl (“Turbo Grignard”);
i-PrMgBr; and
sec-BuMgCl·LiCl.

Based on the screen, all of the above Grignard reagents showed full consumption of Compound A6 by HPLC-UV (i.e., no residual starting material detected) after 0.5 h to 1 h at about 0° C. with about 1.05 to 1.11 equivalents of the Grignard reagent preferred.

### Coupling Reaction

A screen to investigate the coupling with Boc-nortropinone was undertaken, which included p-tolylmagnesium bromide. A screen of seven reactions with different additives (reagents and equivalents) was then performed at room temperature, including:
additives: CeCl₃, LaCl₃·2LiCl, MnCl₂; and
equivalents: 1.5, 2.0.

LaCl₃ showed conversion independently from the equivalents used.

### SUMMARY

The halogen-metal exchange reaction of Compound A6 worked well with different Grignard reagents. The coupling with Boc-nortropinone used LaCl₃·2LiCl to provide good conversion to Compound A8.

The next step was to combine the two steps, and to investigate the coupling of Boc-nortropinone with Compound A6 using different Grignard reagent/additive combinations.

### Screening of Grignard Reagent/Additive Combinations

A summary of the various screening conditions is provided in the Table below:

| | | | | | | | Crude ratio | |
|---|---|---|---|---|---|---|---|---|
| | | | | | | Reaction | Compound | % yield of |
| Experiment | Compound | Compound | | Reagent | Additives | temperature | A7:Compound | purified |
| no. | A7 eq. | A6 eq. | Solvent | (eq.) | (eq.) | (° C.) | A8 | product |
| 1 * | 1.0 | 1.63 | THF | n-BuLi in hexane | — | −95 | 36:64 | 67% |
| | | | anhydrous | (2.2) | | | | |
| 9 | 1.0 | 1.0 | THF | iPrMgBr in THF | CeCl₃ (1.05) | −10 → rt | 95:5 | — |
| | | | anhydrous | (1.11) | | | | |
| 10 | 1.0 | 1.0 | THF | iPrMgBr in THF | LaCl₃ (1.05) | −10 → rt | 30:70 | — |
| | | | anhydrous | (1.11) | | | | |
| 12 | 1.0 | 1.10 | THF | iPrMgBr in THF | LaCl₃ (1.05) | rt | 53:47 | 29% |
| | | | anhydrous | (1.2) | | | | |
| 16 | 1.0 | 1.11 | THF | p-tolylmagnesium | CeCl₃ (1.5) | rt | 47:53 | — |
| | | | anhydrous | bromide (2.0) | | | | |
| 18 | 1.0 | 1.11 | THF | p-tolylmagnesium | LaCl₃ (1.5) | rt | 5:95 | — |
| | | | anhydrous | bromide (2.0) | | | | |
| 19 | 1.0 | 1.11 | THF | p-tolylmagnesium | LaCl₃ (2.0) | rt | 5:95 | — |
| | | | anhydrous | bromide (2.0) | | | | |
| 20 | 1.0 | 1.11 | THF | p-tolylmagnesium | MnCl₂ (1.5) | rt | 26:74 | — |
| | | | anhydrous | bromide (2.0) | | | | |
| 22 | 1.0 | 1.11 | THE | p-tolylmagnesium | — | rt | 46:54 | — |
| | | | anhydrous | bromide (2.0) | | | | |
| 23 | 1.0 | 2.0 | Me—THF | iPrMgBr in | LaCl₃ (1.50) | rt | 44:56 | — |
| | | | anhydrous | Me—THF (2.5) | | | | |
| 24 | 1.0 | 2.0 | Dioxane | iPrMgBr in THF | LaCl₃ (1.50) | rt | 40:60 | — |
| | | | | (2.5) | | | | |
| 29 | 1.0 | 2.0 | THF | iPrMgBr in THF | LaCl₃ (1.50); | rt | 40:60 | — |
| | | | anhydrous | (2.5) | dimethylamino- | | | |
| | | | | | ethylether | | | |
| | | | | | (2.50) | | | |
| 54 | 1.0 | 4.0 | Me—THF | iPrMgBr in | LaCl₃ (1.50) | rt | 36:64 | — |
| | | | anhydrous | Me—THF (4.5) | | | | |
| 55 | 1.0 | 2.0 | Me—THF | iPrMgBr in | LaCl₃ (5.0) | rt | 42:58 | — |
| | | | anhydrous | Me—THF (2.5) | | | | |
| 58 | 1.0 | 2.0 | Me—THF | iPrMgBr in | LaCl₃ (1.5) | rt | 37:63 | — |
| | | | anhydrous | Me—THF (2.5) | | | | |
| 74 | 1.0 | 2.0 | Me—THF | iPrMgBr in | LaCl₃ (0.5) | −20 → −20 | 93:7 | — |
| | | | anhydrous | Me—THF (1.2) | | | | |
| 75 | 1.0 | 2.0 | Me—THF | iPrMgBr in | LaCl₃ (1.0) | 0 → 0 | 41:59 | — |
| | | | anhydrous | Me—THF (1.2) | | | | |
| 77 | 1.0 | 2.0 | Me—THF | iPrMgBr in | LaCl₃ (0.5) | 0 → rt | 27:73 | — |
| | | | anhydrous | Me—THF (1.5) | | | | |
| 81 | 1.0 | 2.0 | Me—THF | iPrMgBr in | LaCl₃ (1.0) | 0 → rt | 19:81 | — |
| | | | anhydrous | Me—THF (1.8) | | | | |
| 104 | 1.0 | 1.5 | Me—THF | iPrMgBr in | LaCl₃ (1.5) | −20 → 0 | 38:62 | 45% |
| | | | anhydrous | Me—THF (1.7) | | | | |
| 105 | 1.0 | 1.5 | Me—THF | iPrMgBr in | LaCl₃ (1.5) | −20 → 0 | 40:60 | 48% |
| | | | anhydrous | Me—THF (1.3) | | | | |

* comparative example

### Overall Results

Based on the experiments, it was determined that the combination of i-PrMgBr and LaCl₃ was preferred for the synthesis of Compound A8. Dioxane and Me-THF also showed goof reaction results.

### Screening of Addition Order, Addition Time, and Stirring Time

A further screen was conducted to assess the effect of the order of reagent addition on the reaction, as follows.

Order of addition of different reagents/reagent mixtures at RT (addition time 1 h). Listed is the last reagent added to the mixture:
addition of nortropinone (all other reagents already present);
addition of Compound A6+i-PrMgBr+LaCl₃;
addition of nortropinone+LaCl₃; and
addition of Compound A6+i-PrMgBr.

Three special addition orders at RT and −78° C.:
Compound A6→LaCl₃→i-PrMgBr→nortropinone;
i-PrMgBr→LaCl₃→Compound A6→nortropinone; and
one-pot reaction (at RT).

Reaction time of Compound A6+i-PrMgBr (with and without LaCl₃):
30 min; and
16 h.

The screening with 1 h addition time showed no significant differences, the conversion was between 54% and 64% Compound A8. The first two tested orders of addition were strongly exothermic and led to only 30-35% conversion with very low IPC HPLC purity at RT of 11.6% and 24.0%. At −78° C., the reactions did not take place. Only when warming up to RT a reaction occurred with conversions of 61% to 73%. Even though the conversion looked promising, the safety risk was considered to be too high to perform this on bigger scale because of accumulation. The one-pot reaction (addition of i-PrMgBr as last reagent) contained various by-products.

The reaction time after the addition of the Grignard reagent i-PrMgBr to Compound A6 was investigated. The reactions with 30 min reaction time before addition to the Boc-nortropinone showed better conversion than the reactions with 16 h reaction time. The LaCl₃ also had an effect on conversion and purity. The reactions where LaCl₃ is present in the reaction mixture from the beginning performed poorer than the reactions where LaCl₃ is added simultaneously with or directly before the nortropinone. Therefore, LaCl₃ should be added either shortly before the nortropinone or at the same time.

### Screening of Equivalents and Temperatures

At first, an initial screening was performed to investigate the influence of different equivalents of LaCl₃ (0.2/1.5/2.0/2.5) at RT. The best results related to conversion and purity were obtained with 1.5 equivalents of LaCl₃. Otherwise, 2.0 equivalents of LaCl₃ showed a slightly better conversion, but the purity was poorer.

The next step was a screening of four different parameters each with three different set points, resulting in nine reactions overall, to determine the best conditions:
equivalents of i-PrMgBr: 1.2/1.5/1.8;
equivalents of LaCl₃: 0.5/1.0/1.5;
temperature halogen-metal exchange: −20° C./0° C./RT; and
temperature reaction: −20° C./0° C./RT.

The results show that the following parameters describe the best conditions:
i-PrMgBr: 1.5 eq.;
LaCl₃: 1.5 eq.;
temperature H-M-exchange: −20° C.; and
temperature reaction: 0° C.

Verification reaction: the optimised conditions were used to perform a verification run with 2.0 g of Boc-nortropinone.

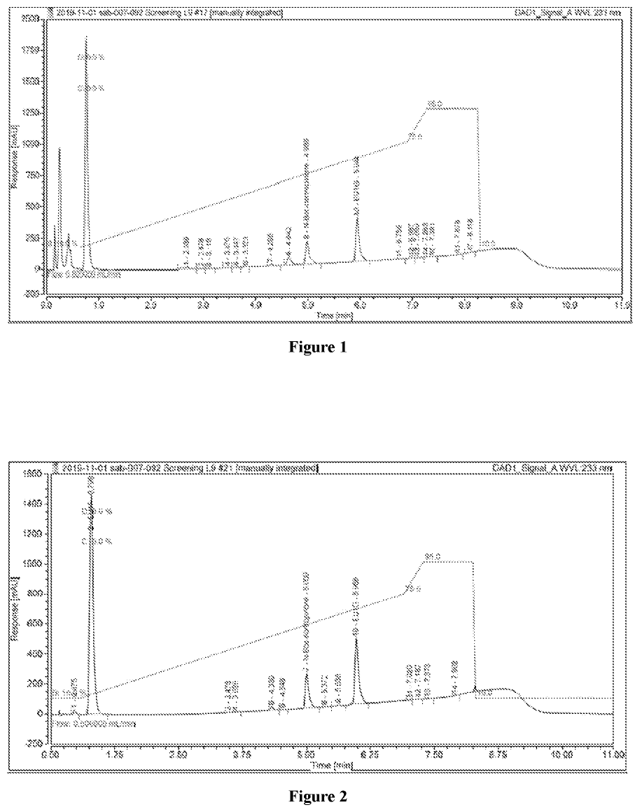

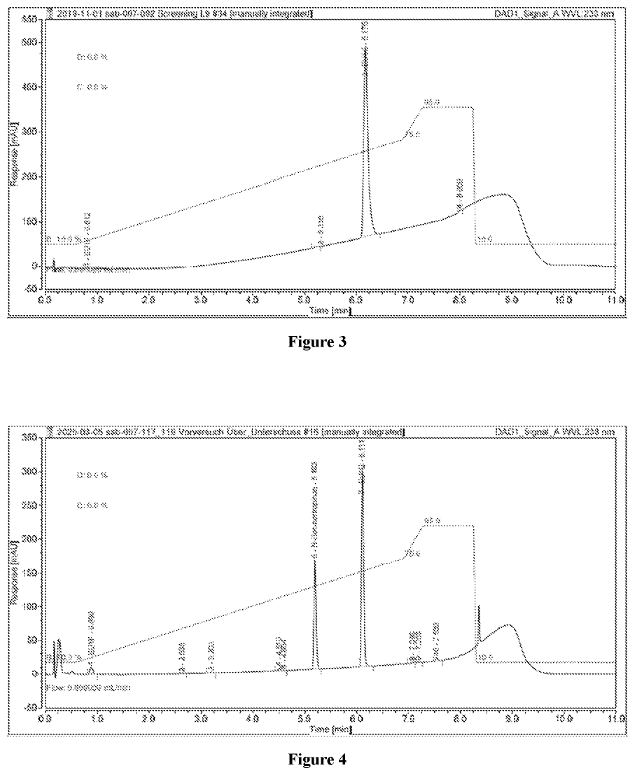

Compound A6 was charged, diluted with 2-Me-THF and cooled to −20° C. i-PrMgBr was added at −20 to −15° C. resulting in a yellow suspension. After stirring for 30 min, the mixture was heated to 0° C. At 0-5° C., a solution of Boc-nortropinone and LaCl₃ in THF was added dropwise within 30 min IPC after 1.5 h showed 35% Boc-nortropinone/65% Compound A8 and an IPC HPLC purity of 51.2% (see FIG. 1). The reaction was quenched with an aqueous solution of citric acid (5%), extracted with 2-Me-THF and the organic phase was washed with an aqueous sodium chloride solution (5%). The organic phase was evaporated to dryness to obtain 4.1 g of crude product with HPLC-assay of 31.0% and HPLC purity of 19.4 area % (9.7 area % nortropinone and 68.1% Compound A6 left) (see FIG. 2). Crystallization from heptanes gave 0.55 g (yield: 20.1%) pure product with HPLC purity of 99.3 area % (see FIG. 3, HPLC chromatogram of purified product).

### Screening of Scale-Up Reaction

For the majority of the previous tests, 2.0 equivalents of Compound A6 were used to ensure a complete conversion of the available (not deprotonated) Boc-nortropinone. As Compound A6 is an expensive starting material, it was decided to test the reaction with lower amounts of Compound A6 (1.5 eq). Furthermore, it was examined whether an excess or a deficit of i-PrMgBr (relative to Compound A6) is better for a scale-up reaction: two experiments were performed on a 2.0 g scale. Reaction conditions were similar to the reaction described above (1.0 eq nortropinone/1.5 eq LaCl₃/−20° C. to 0° C.). The differences were the equivalent of i-PrMgBr (1.7 eq and 1.3 eq, respectively, as opposed to 2.0 eq) and Compound A6 (changed from 2.0 eq to 1.5 eq.)

In the first experiment, an excess of i-PrMgBr (1.7 eq.) was used. IPC showed a Compound A6 to nortropinone ratio of 62.4% to 37.6% with a HPLC purity of 37.3 area % after stirring over night at 0° C. (after 2 h the purity was 43.3%=>degradation over night because of side reactions with the residual i-PrMgBr). After work up, 5.86 g crude product was obtained with an assay of 20.9% (by qNMR) and a HPLC-purity of 60.7 area % Compound A8 (32.7% nortropinone and 3.2% Compound A6). The calculated assay corrected maximum yield is (see FIG. 4: HPLC chromatogram crude, and FIG. 5: quant. NMR).

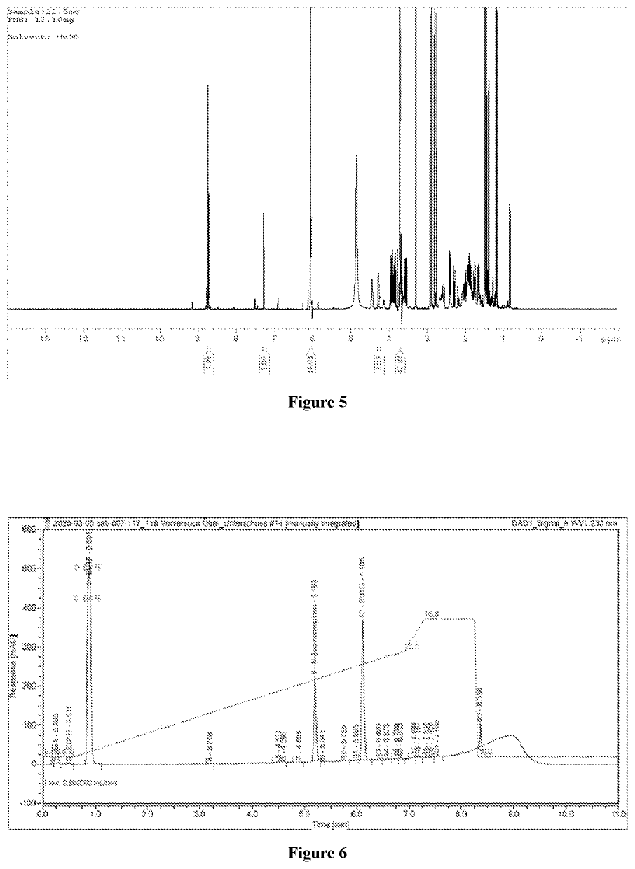

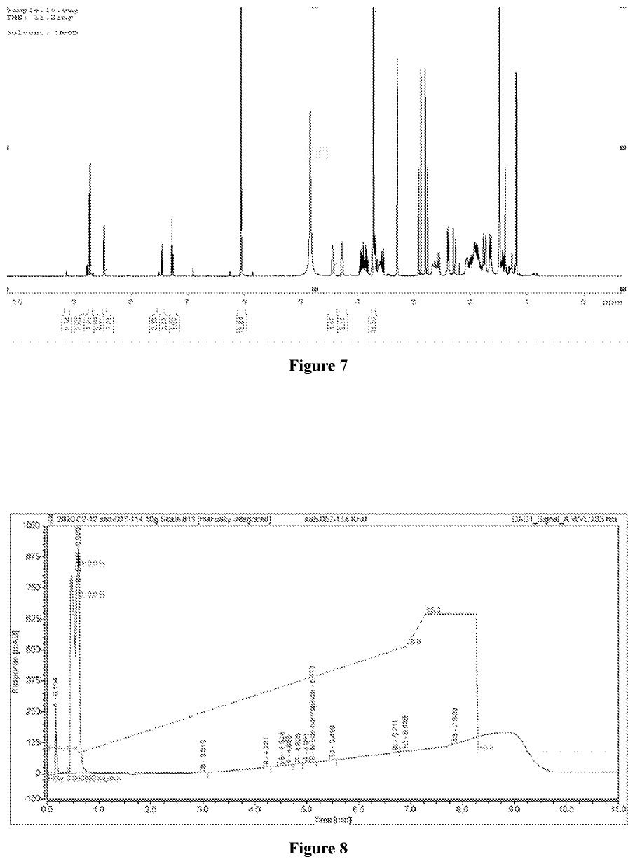

In the second experiment, a deficit of i-PrMgBr (1.3 eq.) was used. IPC showed Compound A6 to nortropinone ratio of 60% to 40% with a HPLC purity of 44.9 area % after stirring over night at 0° C. (Compound A6 not integrated because of excess). 5.47 g crude product was obtained with an assay of 24.0% (by NMR) and a HPLC-purity of 25.2 area % Compound A8 (14.6% nortropinone and 52.1% Compound A6). The calculated assay corrected maximum yield is 48.3% (see FIG. 6: HPLC chromatogram crude, and FIG. 7: NMR crude).

Based on these results, the scale-up was performed with about 1.3 eq of i-PrMgBr to prevent degradation after “full” conversion. Lowering the equivalents of Compound A6 had no negative impact on conversion, but a positive on the price of the manufacturing (less Compound A6 required), so this was also implemented.

### Implementation of Scale-Up Conditions

Compound A6 was charged, diluted with 2-Me-THF and cooled to −20° C. i-PrMgBr was added at −20 to −15° C., resulting in a yellow suspension. After stirring for 30 min, the mixture was heated to 0° C. At 0-5° C., a solution of Boc-nortropinone and LaCl₃ in THF was added dropwise within 30 min IPC after 2.5 h showed 47% Boc-nortropinone/53% Compound A8 and an IPC HPLC purity of 42.9. The reaction was quenched with an aqueous solution of citric acid (5%), extracted with 2-Me-THF and the organic phase was washed with an aqueous sodium chloride solution (5%). The organic phase was split into two parts of similar size. The organic phases were evaporated to dryness to obtain 24.5 g respectively 25.0 g of crude product with NMR-assay of 35.9% respectively 34.6% (assay corrected yields: 43.2%/42.6%) and HPLC purity of 24.6 area % (21.2 area % nortropinone and 52.3% A6 left).

Purification of Compound A8: Compound A8 was purified by chromatography with a heptanes/EtOAc gradient (yield: 33%, purity: 93.8 area %) and crystallized from heptanes (overall yield: 28%, purity 97.2%).

### Summary of the Development of the Grignard Route

In total, more than 100 reactions were performed to develop the alternative Grignard route. Finally, reaction conditions were developed which show quite similar reaction profile and yields as the literature BuLi process, but without needing cryogenic temperatures.

For scale-up, the process without chromatography is preferred (i.e., telescope of Compound A8 into synthesis of Compound A9). To avoid the formation of the new impurity formed during scale-up, the iodopyrimidine was removed by extraction or derivatization.

To avoid purification difficulties, it was decided to telescope the crude Compound A8 to Compound A9 and to do purification after this step.

### Example 5: Synthesis of Compound A9

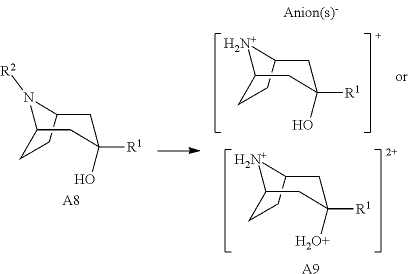

### Scale-Up Telescoped Reaction

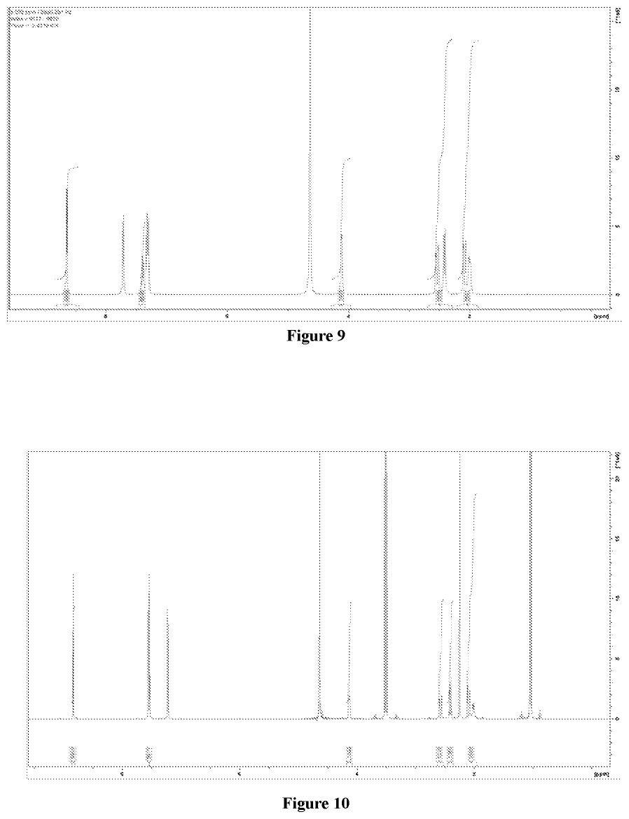

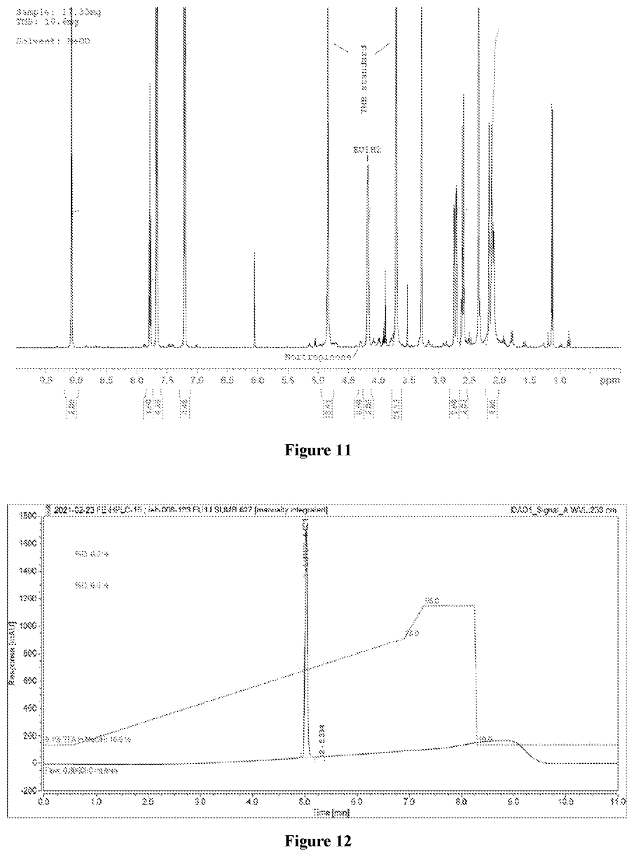

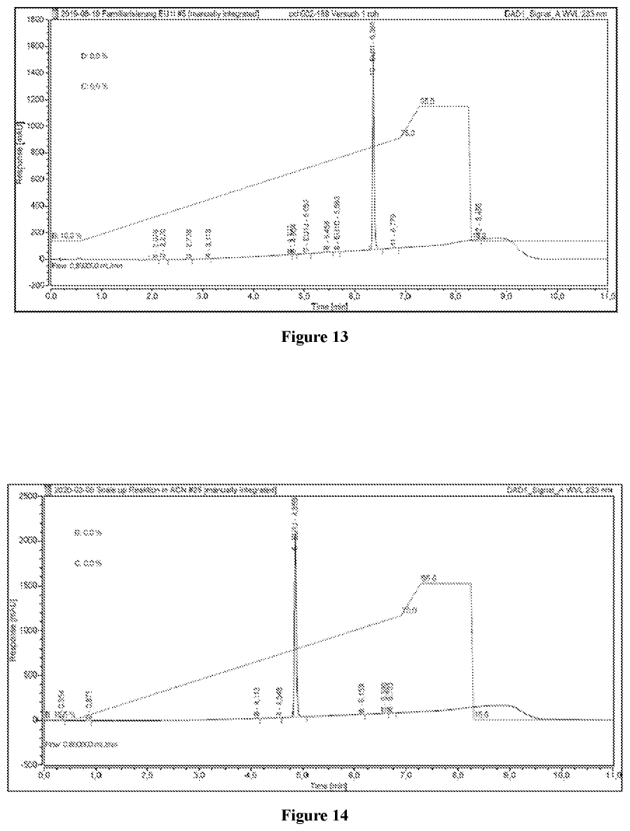

Crude Compound A8 was telescoped into the Boc-cleavage reaction. Sulphonic acid was also used for Boc-deprotection and to generate a stable deprotected salt compound. The crude Compound A8 (50 g) was dissolved in an aqueous solution of 4-toluenesulfonic acid (p-TSA or TsOH) monohydrate (0.5 M, 3.5 eq.). The mixture was heated to 50° C. and stirred for 1-2 h. After IPC showed complete consumption of Compound A8 in the supernatant, the turbid mixture was allowed to cool to r.t. The resulting precipitate was filtered off and rinsed with MeTHF. After drying in vacuo at r.t. the pTSA salt of Compound A9 was obtained as a colorless to off-white solid (36.9% yield at >99% purity, see FIG. 9). The salt was also identified to reveal a 2:1 composition of pTSA:Compound A9. The NMR assay of the salt was 99.6% purity.

### Example 6: Synthesis of Compound 1

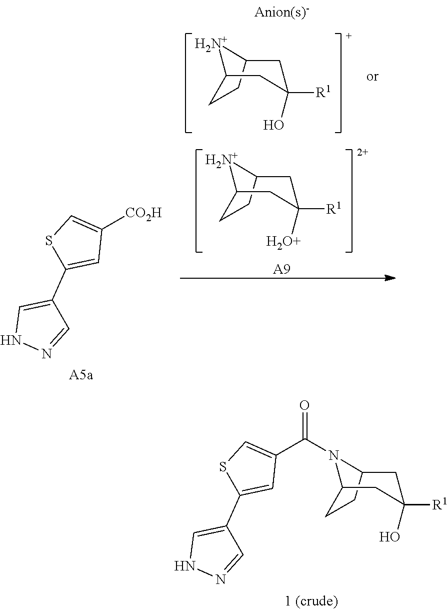

To a suspension of Compound A5a (1.00 eq.), Compound A9 (EU1H2*2 pTSA (1.10 eq.), and HOPO (1.50 eq.) in acetonitrile/water (1:1 v/v, 31.0 v/w) was added DIPEA (3.5 eq.) and the resulting mixture stirred for 5 min DIC (1.50 eq.) was added, the mixture heated to 60° C. and stirred until complete consumption of EU1D2 was observed (6-19 h). Acetonitrile was distilled off and the mixture allowed to cool to r.t. It was acidified by slowly adding 2 M HCl (1.0 eq.) and the aq. layer washed with iPrOAc (3×21.3 v/w). To the aq. layer was added EtOH (7.5 v/w) and the mixture heated to 45° C. NaOH (30%, 1.00 eq.) was added dropwise until pH 12. Seed was used for purification of the product before drying to remove solvent. After evaporation under reduced pressure Compound 1 was obtained as off white solid (20.1 g, 82.4%) with a purity of 99.7 area % (see FIG. 12).

Oxymapure and EDC were also used for the amide coupling reaction. 6.5 g of Compound A5 was dissolved in 13 vol. eq. of acetonitrile. Oxymapure was added and suspension was cooled down to −10° C. EDC×HCl was added and mixture was allowed to stir for 30 min. DIPEA and Compound A9 were then added. Mixture was allowed to warm up to room temperature. Mixture became a solution over time. After completion of the reaction, half of the reaction mixture was taken to test a proposed aqueous work up. Work up: Reaction mixture was added dropwise to three times the amount of water to acetonitrile to give a light suspension. Solid sodium carbonate was added until pH 9-12. Solvents are then removed under reduced pressure and chased twice with 2 vol.eq of water. Solid was suspended in water and 60% H₂SO₄ was added until pH 0. Solution was then washed twice with Me-THF to remove coupling reagents. Concentrated sodium hydroxide solution was added until pH 11. Mixture was heated up to 50° C. and saturated with Na₂SO₄. Mixture was then extracted twice with a Me-THF/EtOH (3/1) mixture. Organic layer was then dried under reduced pressure to afford an orange solid as the crude product. HPLC: 97.71% Area. qNMR: 41.47%. Analysis of the reaction mixture by NMR showed that 79% of the theoretical amount of Compound 1 was in the acetonitrile solution. Crude Compound 1 was suspended in 6 vol. eq. of EtOH/H₂O 1/1 and heated up to 82° C. The mixture was then cooled down to 0° C. before being filtered. Solid was then stirred with 2 vol. eq. of water for 30 min at 0° C., before being once again filtered. Solids were dried at 50° C. under reduced pressure to afford 3.24 g of a grey solid as pure Compound 1. HPLC Area %: 98.53%. qNMR: 97.67% (see FIG. 14).

1. A process for preparing an aza-bicyclic compound of Formula 4: 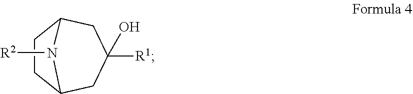
comprising a Grignard reaction of a nortropinone compound of Formula 5: 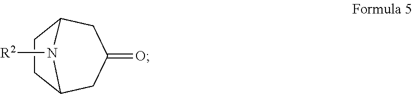
with a halogenated compound of Formula 6:
X—R¹ Formula 6;
wherein
R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, —C₁₋₆haloalkyl, —O—C₁₋₆haloalkyl, —CN, —NR³R⁴, —COR³, —CO₂R³, and each R³ and R⁴ are independently selected from the group consisting of hydrogen and —C₁₋₆alkyl;
R² is an amine protecting group; and
X is a halogen.

2. The process of claim 1, wherein R¹ is a monocyclic or bicyclic heteroaryl group each unsubstituted or substituted with one or more substituents selected from the group consisting of halogen, —OH, —C₁₋₆alkyl, —O—C₁₋₆alkyl, C₁₋₆haloalkyl, and —O—C₁₋₆haloalkyl.

3. The process of claim 1, wherein R¹ is pyrimidine.

4. The process of claim 1, wherein R² is an amine protecting group selected from the group consisting of carbamate, amide, benzyl, benzylidene, tosyl, and trityl.

5. The process of claim 1, wherein R² is a tert-butyloxycarbonyl (BOC) group.

6. (canceled)

7. The process of claim 1, wherein the Grignard reaction comprises the steps of i) a halogen-metal exchange reaction including iPrMgBr and ii) a coupling reaction including LaCl₃.

8. (canceled)

9. The process of claim 1, wherein the aza-bicyclic compound of Formula 4 is a protected amine compound of Formula 4a: 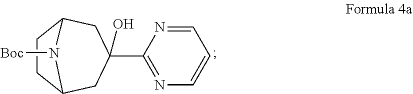 [SMILES: CC(C)(C)OC(=O)N1C2CCC1CC(O)(c1ncccn1)C2]
and the process comprises reacting a tropinone compound of Formula 5a: 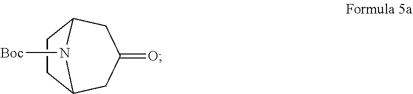 [SMILES: CC(C)(C)OC(=O)N1C2CCC1CC(=O)C2]
with a halogenated compound of Formula 6a:

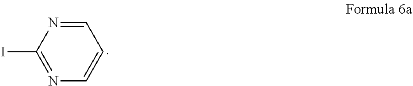

10. A process for preparing a salt of an amine bicyclic compound of Formula 3: 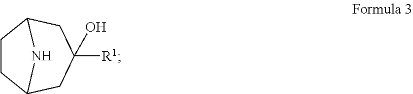
wherein the process comprises using a sulphonic acid to remove an amine protecting group from an aza-bicyclic compound of Formula 4 prepared according to claim 1, and forming a sulphonate salt thereof.

11. The process of claim 10, wherein the salification comprises 4-toluenesulphonic acid (p-TSA) to prepare a p-TSA salt of Formula 3.

12. (canceled)

13. A process for preparing a heterocyclic methanone compound of Formula 1: 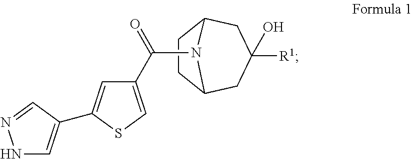
comprising reacting a carboxylic acid compound of Formula 2 or a salt thereof: 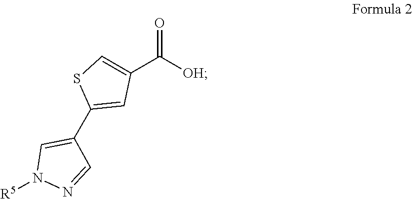
with an amine bicyclic compound of Formula 3 or a salt thereof, in the presence of at least one coupling reagent selected from an oxime coupling reagent and a carbodiimide coupling reagent: 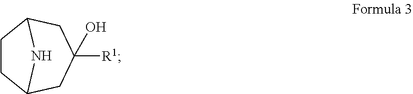
wherein
R¹ is selected from a carbocyclyl or heterocyclyl, wherein each carbocyclyl and heterocyclyl is a monocyclic or bicyclic group each unsubstituted or substituted with one or more substituents selected from the group consisting of halogen, —OH, —C₁₋₆alkyl, —O—C₁₋₆alkyl, C₁₋₆haloalkyl, —O—C₁₋₆haloalkyl, —CN, —NR³R⁴, —COR³, —CO₂R³, and each R³ and R⁴ are independently selected from the group consisting of hydrogen and C₁₋₆alkyl;
R⁵ is hydrogen or an amine protecting group.

14-16. (canceled)

17. The process of claim 13, wherein the coupling reagent is a carbodiimide coupling reagent selected from DIC (diisopropylcarbodiimide).

18. The process of claim 13, further comprising one or more additives, wherein the additive is selected from an N-oxide reagent and a base, and wherein the N-oxide reagent is 2-hydroxypyridine-N-oxide (HOPO) and the base is N,N-diisopropylethylamine (DIPEA).

19-22. (canceled)

23. The process of claim 13, wherein R¹ is a monocyclic or bicyclic heteroaryl group each unsubstituted or substituted with one or more substituents selected from the group consisting of halogen, —OH, —C₁₋₆alkyl, —O—C₁₋₆alkyl, C₁₋₆haloalkyl, and —O—C₁₋₆haloalkyl.

24. The process of claim 13, wherein R¹ is pyrimidine.

25. The process of claim 13, wherein R⁵ is an amine protecting group THP or hydrogen.

26. (canceled)

27. The process of claim 13, wherein the compound of Formula 3 is a sulphonate salt.

28-30. (canceled)

31. The process of claim 13, wherein the heterocyclic methanone compound of Formula 1 is a compound of Formula 1a: 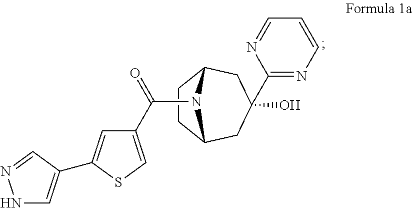 [SMILES: O=C(c1csc(-c2cn[nH]c2)c1)N1[C@@H]2CC[C@H]1C[C@](O)(c1ncccn1)C2]
wherein the process comprises reacting a carboxylic acid compound of Formula 2a or salt thereof: 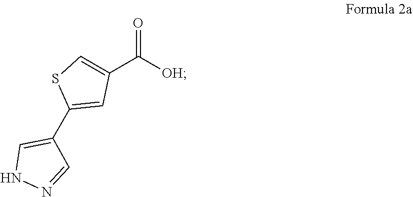 [SMILES: O=C(O)c1csc(-c2cn[nH]c2)c1]
with a sulphonate salt of Formula 3a in the presence of a carbodiimide coupling reagent: 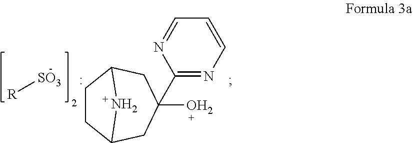
wherein R is selected from an alkyl, aryl and alkyl aryl, each of which are optionally substituted.

32. The process of claim 13, wherein the carboxylic acid compound of Formula 2 is prepared by saponification with a base of an ester compound of Formula 7: 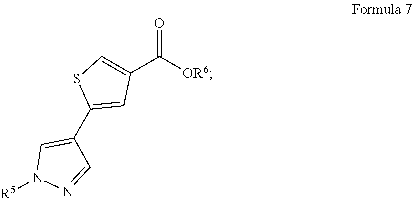
wherein R⁵ is hydrogen or an amine protecting group and R⁶ is an ester protecting group.

33-35. (canceled)

36. A compound of Formula 1 or Formula 1a: 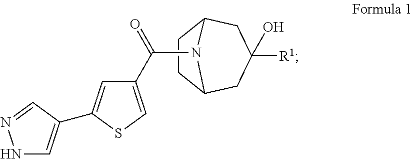
prepared by the process of claim 13.

37. A composition comprising a compound of Formula 1a: 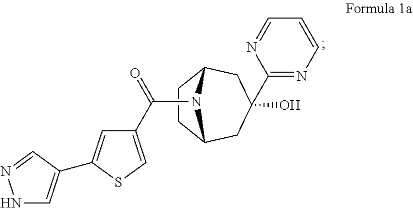 [SMILES: O=C(c1csc(-c2cn[nH]c2)c1)N1[C@@H]2CC[C@H]1C[C@](O)(c1ncccn1)C2]
wherein any impurities, if present, are in an amount (by weight % of the total composition) of less than about 1 wt %.
